(19) United States
(12) Patent Application Publication
(10) Pub. No.: US 2022/0056012 A1
(43) Pub. Date: Feb. 24, 2022

(54) AMORPHOUS FORM OF A MALT1 INHIBITOR AND FORMULATIONS THEREOF

(71) Applicant: Janssen Pharmaceutica NV, Beerse (BE)

(72) Inventors: Kristof Leonard KIMPE, Beerse (BE); Sune Klint ANDERSEN, Geel (BE); Matthieu Jean M RAVELINGIEN, Geel (BE); Tom Els R HUYBRECHTS, Merelbeke (BE)

(21) Appl. No.: 17/407,208

(22) Filed: Aug. 20, 2021

(30) Foreign Application Priority Data
Aug. 21, 2020 (EP) 20192098.0

(51) Int. Cl. C07D 401/14; A61K 9/20; A61K 9/28; A61K 47/32; A61K 47/38; A61K 9/48; A61K 9/16; B29B 9/06; B29C 43/00

(52) U.S. Cl. CPC C07D 401/14; B29K 2105/0035; A61K 9/2009; A61K 9/2013; A61K 9/28; A61K 47/32; A61K 47/38; A61K 9/4866; A61K 9/485; A61K 9/1682; A61K 9/4833; A61K 9/2095; B29B 9/06; B29C 43/003; C07B 2200/13; A61K 9/2054

(57) ABSTRACT

The present invention relates to the amorphous form of a MALT1 inhibitor, methods of preparation thereof and pharmaceutical compositions comprising this amorphous form.

### FIELD OF THE INVENTION

The technical field of the present invention is in pharmaceuticals, particularly formulations of a drug in solid form.

### BACKGROUND OF THE INVENTION

Many active pharmaceutical ingredients (API) have properties such as hydrophobicity and instability leading to challenges in providing suitable pharmaceutical formulations.

MALT1 (mucosa-associated lymphoid tissue lymphoma translocation 1) is a key mediator of the classical NF-κB signaling pathway. Patent publication WO 2018/119036 discloses a class of active pharmaceutical agents which are MALT1 inhibitors that may provide a therapeutic benefit to patients suffering from cancer and/or immunological diseases.

There exists a need for improved pharmaceutical formulations of active pharmaceutical ingredients (API), such as the MALT1 inhibitors described in WO 2018/119036. In particular there exists a need for pharmaceutical formulations with an acceptable bioavailability, in particular in a solid dosage form.

1-(1-oxo-1,2-dihydroisoquinolin-5-yl)-5-(trifluoromethyl)-N-[2-(trifluoromethyl)pyridin-4-yl]-1H-pyrazole-4-carboxamide has the following structure, and is referred herein as Compound A, or compound of formula A:

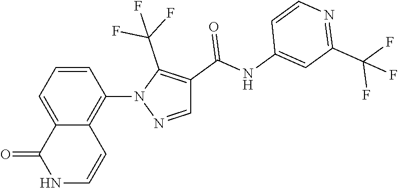 [SMILES: O=C(Nc1ccnc(C(F)(F)F)c1)c1cnn(-c2cccc3c(=O)[nH]ccc23)c1C(F)(F)F]

Compound A may be prepared, for example, according to the procedure as described in Example 158 of WO 2018/119036, which is incorporated herein by reference. The procedure of Example 158 has been determined as providing crystalline Form I of Compound A hydrate. Form I exhibits hygroscopic behavior. Form I has an X-ray powder diffraction pattern comprising peaks at 8.4, 12.7, 13.3, and 16.7 degrees two theta ±0.2 degrees two theta. The X-ray powder diffraction pattern may further comprise at least one peak selected from 6.7, 10.0, 10.7, 12.0, 12.3, 13.5, 14.1, 14.6, 15.4, 15.6, 16.0, 18.1, 18.4, 19.2, 20.0, 20.3, 21.1, 22.0 and 24.9 degrees two theta ±0.2 degrees two theta. Form I may also be characterized by a differential scanning calorimetry (DSC) thermogram comprising an endotherm with an onset temperature of 66° C. and a peak temperature at 99° C. The DSC may comprise a second endotherm with onset temperature of 145° C. and a peak temperature of 157° C.

Another crystalline form (Form III) of compound A monohydrate may be prepared according to the procedures described in Examples 2, 3, and 3b of WO2020/169736, which is incorporated herein by reference. Form III has an X-ray powder diffraction pattern comprising peaks at 16.4, 23.7 and 25.7 degrees two theta ±0.2 degrees two theta. The X-ray powder diffraction pattern may further comprise at least one peak selected from 13.6, 17.9, 22.6, 24.5, 25.2, and 27.1 degrees two theta ±0.2 degrees two theta. The X-ray powder diffraction pattern may further comprise at least one peak selected from 8.3, 8.6, 11.5, 14.0, 15.4, 17.5, 19.7, 22.0, 22.2, 24.0, and 29.9 degrees two theta ±0.2 degrees two theta. Form III may also be characterized by a DSC comprising an endotherm with an onset temperature of about 142° C. and a peak temperature at about 158° C.

WO2020/169738 describes polyethylene glycol (PEG) based formulations comprising Compound A.

### Objectives

An objective of the present invention is to provide an isolated amorphous form of compound A, or a pharmaceutically acceptable salt form thereof.

An objective of the present invention is to provide a solid-state form of compound A, or a pharmaceutically acceptable salt form thereof, that is kinetically stable according to regulatory requirements.

An objective of the present invention is to improve the solubility of compound A, or a pharmaceutically acceptable salt form thereof.

An objective of the present invention is to improve the solubility of compound A, or a pharmaceutically acceptable salt form thereof, in an aqueous solution.

An objective of the present invention is to improve the solubility of compound A, or a pharmaceutically acceptable salt form thereof, in gastrointestinal media.

An objective of the present invention is to improve the dissolution rate of compound A, or a pharmaceutically acceptable salt form thereof.

An objective of the present invention is to improve the permeability of compound A, or a pharmaceutically acceptable salt form thereof, across biological membranes.

An objective of the present invention is to improve the oral absorption of compound A, or a pharmaceutically acceptable salt form thereof.

An objective of the present invention is to improve the bioavailability of compound A, or a pharmaceutically acceptable salt form thereof.

An objective of the present invention is to improve the shelf-life of compound A, or a pharmaceutically acceptable salt form thereof.

An objective of the present invention is to provide a solid-state form of compound A, or a pharmaceutically acceptable salt form thereof, with a shelf-life of at least 1 year, at least 3 years, or up to 5 years.

An objective of the present invention is to provide a solid-state form of compound A, or a pharmaceutically acceptable salt form thereof, with a shelf-life of 6 months under accelerated conditions (40° C./75 RH).

An objective of the present invention is to improve the physical stability of amorphous solid dispersions (ASDs) of compound A, or a pharmaceutically acceptable salt form thereof.

An objective of the present invention is to improve the kinetic stability of ASDs of compound A, or a pharmaceutically acceptable salt form thereof, during its shelf-life.

An objective of the present invention is to increase the drug load of solid dosage forms of compound A, or a pharmaceutically acceptable salt form thereof.

An objective of the present invention is to reduce the amount of excipients in solid dosage forms of compound A, or a pharmaceutically acceptable salt form thereof.

An objective of the present invention is to provide formulations of compound A, or a pharmaceutically acceptable salt form thereof, that are directly compressible into tablets.

An objective of the present invention is to reduce the food effect on the bioavailability of compound A comprised in tablets, or a pharmaceutically acceptable salt form thereof.

An objective of the present invention is to reduce the pill burden of cancer patients treated with compound A, or a pharmaceutically acceptable salt form thereof.

An objective of the present invention is to improve the therapy adherence of a cancer patient treated with compound A, or a pharmaceutically acceptable salt form thereof.

An objective of the present invention is to improve the therapy efficiency of a cancer patient treated with compound A, or a pharmaceutically acceptable salt form thereof.

It has been unexpectedly found that the amorphous form of Compound A and amorphous solid dispersions thereof are exceptionally physically stable with a glass transition temperature of 133° C. The amorphous form of Compound A form belongs to GFA (glass forming ability) class III, indicating good physical stability, as evidenced by stability DSC data (after 6 MOS at 40°/75% RH, the sample is still amorphous with open-dish).

This stability was not expected when departing from the melting points of the hydrate forms.

Crystalline forms are usually more physically stable than amorphous forms. The amorphous form of the present invention while being surprisingly more physically stable, it is still soluble.

In addition, mixtures of amorphous and crystalline forms of Compound A as claimed, of more than 90% w/w in amorphous form, may still show acceptable dissolution rates.

Furthermore, when preparing amorphous solid dispersions of Compound A, the prejudice was that there would be more likelihood of incompatibility with any of the polymers, but again, surprisingly, this incompatibility did not occur.

Surprisingly as well, the dissolution rates in a FaSSIF medium dropped considerably when using crystalline form I of compound A, whilst with different amorphous preparations, the dissolution rates were much better, cfr. FIG. 20.

In addition, the inventors managed to prepare ASDs with high API/polymer ratios, which is convenient to increase drug load, to decrease pill burden for the patient and tablet dimensions. As the person skilled in the art knows, high API/polymer ratios are prone to crash-out and are more difficult to formulate into a tablet. ASDs with high API/polymer ratios show lower tabletability; i.e., they are less porous and more compact, and that requires higher compression forces during tableting.

### SUMMARY OF THE INVENTION

The present invention relates to an isolated, 1 (1 oxo-1,2 dihydroisoquinolin-5 yl)-5 (trifluoromethyl)-N-[2 (trifluoromethyl)pyridin-4 yl]-1H-pyrazole-4 carboxamide (compound A), or a pharmaceutically acceptable salt form thereof, in amorphous form or non-crystalline phase, wherein the amorphous form or non-crystalline phase of compound A is present in a weight percentage in respect of any crystalline form of compound A, of more than 90% w/w, preferably at least 95% w/w.

The present invention relates to an amorphous solid dispersion comprising compound A, or a pharmaceutically acceptable salt form thereof; and an orally pharmaceutically acceptable polymer.

In the solid dispersion, the weight-by-weight ratio of (compound A):(orally pharmaceutically acceptable polymer) may be in the range of 5:1 to 1:100; 2:1 to 1:10; 2:1 to 1:5; 1:3; 1:2; 1:1; 2:1; 3:1; 5:1.

In the amorphous solid dispersion, the weight-by-weight ratio of (compound A):(orally pharmaceutically acceptable polymer) may be in the range of 5:1 to 1:5; preferably in the ratios of 5:1, 4:1, 3:1, 2:1, 1:1, 1:2, 1:3, 1:4, and 1:5. In another embodiment, the weight-by-weight ratio of (compound A):(orally pharmaceutically acceptable polymer) is 1:1 or 1:2. In another embodiment, the weight-by-weight ratio of (compound A):(orally pharmaceutically acceptable polymer) is 1:2.

In another embodiment, the weight-by-weight ratio of (compound A):(orally pharmaceutically acceptable polymer) is in the range of 1.1:2 to 0.9:2.

In another embodiment, the weight-by-weight ratio of (compound A):(orally pharmaceutically acceptable polymer) is in the range of 1.1:1 to 0.9:1.

In another embodiment, the weight-by-weight ratio of (compound A):(orally pharmaceutically acceptable polymer) is in the range of 1.1:1 to 0.9:2.

In the amorphous solid dispersion, the orally pharmaceutically acceptable polymer may be a polymer used for spray-drying that has an apparent viscosity when dissolved at 20° C. of 1 to 5000 mPa·s, of 1 to 500 mPa·s, or of 1 to 100 mPa·s; or the orally pharmaceutically acceptable polymer has an apparent viscosity in an organic solvent of 1 to 5000 mPa·s, of 1 to 500 mPa·s, or of 1 to 100 mPa; or the orally pharmaceutically acceptable polymer may be a polymer used for Hot Melt Extrusion and the molten polymer has an apparent viscosity of 1 to 1,000,000 Pa·s, of 100 to 100,000 Pa·s, or of 500 to 10,000 Pa·s.

In the amorphous solid dispersion, the orally pharmaceutically acceptable polymer may be selected from the group comprising:
alkylcelluloses such as methylcellulose;
hydroxyalkylcelluloses such as hydroxymethylcellulose, hydroxyethylcellulose, hydroxypropylcellulose and hydroxybutylcellulose;
hydroxyalkyl alkylcelluloses such as hydroxyethyl methylcellulose and hydroxypropyl methylcellulose;
carboxyalkylcelluloses such as carboxymethylcellulose;
alkali metal salts of carboxyalkylcelluloses such as sodium carboxymethylcellulose;
carboxyalkylalkylcelluloses such as carboxymethylethylcellulose;
carboxyalkylcellulose esters;
hydroxypropylmethylcellulose phthalate (HPMCP);
chitin derivates such as chitosan;
polysaccharides such as starches, pectines (sodium carboxymethylamylopectine), cyclodextrins or a derivative thereof, carrageenans, galactomannans, tragacanth, agar agar, gummi arabicum, guar gummi and xanthan gummi;
polyacrylic acids, olyacrylates, and the salts thereof;
polymethacrylic acids, polymethacrylates, the salts and esters thereof, methacrylate copolymers;
polyvinylalcohol (PVA), co-polymers of PVA (e.g., Kollicoat® IR), crospovidone (PVP-CL), polvinylpyrrolidone-polyvinylacetate copolymer (PVP-PVA);
polyalkylene oxides such as polyethylene oxide and polypropylene oxide and copolymers of ethylene oxide and propylene oxide;
polymers of ethylene oxide or polyethylene glycols of molecular weights in the range of 1500-20000, particularly with MW of 4000-6000;
polyvinylpyrrolidone (PVP) of MW ranging from 2500 to 3000000;
Gelita® Collagel; or
any combination thereof;
and optionally a surface-active carrier.

In the amorphous solid dispersion, the orally pharmaceutically acceptable polymer may be selected from the group comprising:
alkylcelluloses such as methylcellulose;
hydroxyalkylcelluloses such as hydroxymethylcellulose, hydroxyethylcellulose, hydroxypropylcellulose and hydroxybutylcellulose;
hydroxyalkyl alkylcelluloses such as hydroxyethyl methylcellulose and hydroxypropyl methylcellulose;
carboxyalkylcelluloses such as carboxymethylcellulose;
alkali metal salts of carboxyalkylcelluloses such as sodium carboxymethylcellulose;
carboxyalkylalkylcelluloses such as carboxymethylethylcellulose;
carboxyalkylcellulose esters;
hydroxypropylmethylcellulose phthalate (HPMCP);
chitin derivates such as chitosan;
polysaccharides such as starches, pectines (sodium carboxymethylamylopectine), cyclodextrins or a derivative thereof, carrageenans, galactomannans, tragacanth, agar agar, gummi arabicum, guar gummi and xanthan gummi;
polyacrylic acids, olyacrylates, and the salts thereof;
polymethacrylic acids, polymethacrylates, the salts and esters thereof, methacrylate copolymers;
polyvinylalcohol (PVA), co-polymers of PVA (e.g., Kollicoat® IR), crospovidone (PVP-CL), polvinylpyrrolidone-polyvinylacetate copolymer (PVP-PVA);
polyalkylene oxides such as polyethylene oxide and polypropylene oxide and copolymers of ethylene oxide and propylene oxide;
polyvinylpyrrolidone (PVP) of MW ranging from 2500 to 3000000;
Gelita® Collagel; or
any combination thereof;
and optionally a surface-active carrier.

In the amorphous solid dispersion, the orally pharmaceutically acceptable polymer may be HPMCAS, HPMC E5, Eudragit® E, Eudragit® L, PVP VA64, any combination thereof; and the orally pharmaceutically acceptable polymer or combination thereof may be optionally mixed with Sodium Lauryl Sulfate (SLS).

In the amorphous solid dispersion, the orally pharmaceutically acceptable polymer may be HPMCAS. In the amorphous solid dispersion, the orally pharmaceutically acceptable polymer may be HPMCAS-LG.

In the solid amorphous dispersion, the HPMCAS may be HPMCAS-LG, HPMCAS-MG, HPMCAS-HG, HPMCAS-LF, HPMCAS-MF, HPMCAS-HF, HPMCAS-LMP, HPMCAS-MMP, HPMCAS-HMP, Affinisol™ HPMCAS 716, Affinisol™ HPMCAS 912, or Affinisol™ HPMCAS 126.

In the amorphous solid dispersion, Eudragit® L may be Eudragit® L 100-55.

In the amorphous solid dispersion, the orally pharmaceutically acceptable polymer may be HPMC E5 mixed with a surface-active carrier, preferably SLS.

The present invention relates to a particle comprising the amorphous solid dispersion as described herein.

The particle comprising the amorphous solid dispersion as described herein, may have a volume weighted particle size distribution Dv50, as measured by a static light scattering instrument, of from about 20 μm to about 90 μm, preferably from about 25 μm to about 80 μm, more preferably from about 25 μm to about 65 μm.

The particle comprising the amorphous solid dispersion as described herein, may have a Dv10 of volume weighted particle size distribution from about 1 μm to about 15 μm; and may have a Dv90 of the volume weighted particle size distribution of from about 40 μm to about 200 μm.

The particle comprising the amorphous solid dispersion as described herein may further comprise a pharmaceutically acceptable carrier.

The present invention relates to a particle comprising compound A in amorphous form or non-crystalline phase, or a pharmaceutically acceptable salt form thereof.

The particle comprising compound A in amorphous form or non-crystalline phase, or a pharmaceutically acceptable salt form thereof, may have a volume weighted particle size distribution Dv50, as measured by a static light scattering instrument, of from about 1 μm to about 100 μm, preferably from about 5 μm to about 80 μm, more preferably from about 25 μm to about 75 μm.

The particle comprising compound A in amorphous form or non-crystalline phase, or a pharmaceutically acceptable salt form thereof, may have a Dv10 of volume weighted particle size distribution from about 0.1 μm to about 15 μm; and a Dv90 of the volume weighted particle size distribution of from about 3 μm to about 250 μm.

The particle comprising compound A in amorphous form or non-crystalline phase, or a pharmaceutically acceptable salt form thereof, may further comprise a pharmaceutically acceptable carrier.

The present invention relates to a pharmaceutical composition comprising a pharmaceutically acceptable carrier; and (i) a therapeutically effective amount of compound A in amorphous form or non-crystalline phase, or a pharmaceutically acceptable salt form thereof; (ii) a therapeutically effective amount of an amorphous solid dispersion comprising compound A, or a pharmaceutically acceptable salt form thereof; and an orally pharmaceutically acceptable polymer, as described herein; (iii) a therapeutically effective amount of the particles comprising the amorphous solid dispersion as described herein; or (iv) a therapeutically effective amount of the particles comprising compound A in amorphous form or non-crystalline phase, or a pharmaceutically acceptable salt form thereof, as described herein.

The pharmaceutical composition as described herein, may be a solid oral dosage form.

The pharmaceutical composition as described herein, may be a tablet, capsule, sachet, pill, lozenge, caplet, capsule, sachet, or troche.

The pharmaceutical composition as described herein, may be a tablet, wherein the pharmaceutically acceptable carrier may comprise a disintegrant, a glidant, a lubricant, a diluent, optionally a wetting agent, optionally a binder, and optionally a coating material.

The pharmaceutical composition as described herein, may be a capsule or a sachet, optionally further comprising a diluent.

The present invention relates to pharmaceutical compositions having the following compositions:

| | Spray Dried Powder: Compound A/Polymer Ratio | | | | | |
|---|---|---|---|---|---|---|
| | 1/2 | | 1/1 | | 2/1 | |
| | mg/tablet | % w/w | mg/tablet | % w/w | mg/tablet | % w/w |
| Spray Dried Powder | 300.00 | 30.00 | 200.00 | 30.00 | 150.00 | 30.00 |
| comprising Compound A and | | | | | | |
| polymer X | | | | | | |
| Microcrystalline cellulose | 367.50 | 36.75 | 245.00 | 36.75 | 183.75 | 36.75 |
| (Avicel PH-101) | | | | | | |
| Croscarmellose Sodium | 25.00 | 2.50 | 16.67 | 2.50 | 12.50 | 2.50 |
| (Ac-Di-Sol SD-711) | | | | | | |
| Silica, Colloidal Anhydrous | 5.00 | 0.50 | 3.33 | 0.50 | 2.50 | 0.50 |
| (Aerosil 200) | | | | | | |
| Magnesium Stearate | 2.50 | 0.25 | 1.67 | 0.25 | 1.25 | 0.25 |
| (Ligamed MF-2-V) | | | | | | |
| Silicified Microcrystalline | 262.50 | 26.25 | 175.00 | 26.25 | 131.25 | 26.25 |
| cellulose | | | | | | |
| (Prosolv SMCC HD 90) | | | | | | |
| Croscarmellose Sodium | 25.00 | 2.50 | 16.67 | 2.50 | 12.50 | 2.50 |
| (Ac-Di-Sol SD-711) | | | | | | |
| Silica, Colloidal Anhydrous | 5.00 | 0.50 | 3.33 | 0.50 | 2.50 | 0.50 |
| (Aerosil 200) | | | | | | |
| Magnesium Stearate | 7.50 | 0.75 | 5.00 | 0.75 | 3.75 | 0.75 |
| (Ligamed MF-2-V) | | | | | | |
| Core Tablet | 1000.00 | 100.00 | 666.67 | 100.00 | 500.00 | 100.00 |

wherein polymer X is HPMCAS-LG or HPMC E5; and
wherein the Core Tablet is optionally coated; preferably coated with coating powder pink
Opadry II 85F250050; more preferably coated with 2-5% (w/w) of coating powder pink
Opadry II 85F250050; more preferably coated with 3% (w/w) of coating powder pink
Opadry II 85F250050. The w/w ratio refers to the content by weight of coating powder
pink Opadry II 85F250050 in respect of the content by weight of the Core Tablet.

The present invention relates to pharmaceutical compositions having the following compositions:

| | 65 mg | | 80 mg | |
|---|---|---|---|---|
| | mg/tablet | % w/w | mg/tablet | % w/w |
| Spray Dried Dispersion Powder | 195.000 | 30.00 | 240.00 | 30.00 |
| comprising a 33.3/66.7 | | | | |
| ratio of Compound A/HPMCAS-LG | | | | |
| Microcrystalline cellulose (Avicel PH-101) | 238.875 | 36.75 | 294.00 | 36.75 |
| Croscarmellose Sodium (Ac-Di-Sol SD-711) | 16.250 | 2.50 | 20.00 | 2.50 |
| Silica, Colloidal Anhydrous (Aerosil 200) | 3.250 | 0.50 | 4.00 | 0.50 |
| Magnesium Stearate (Ligamed MF-2-V) | 1.625 | 0.25 | 2.00 | 0.25 |
| Total Intragranular | 455.000 | 70.00 | 560.00 | 70.00 |
| Silicified Microcrystalline cellulose | 170.625 | 26.25 | 210.00 | 26.25 |
| (Prosolv SMCC HD 90) | | | | |
| Croscarmellose Sodium (Ac-Di-Sol SD-711) | 16.250 | 2.50 | 20.00 | 2.50 |
| Silica, Colloidal Anhydrous (Aerosil 200) | 3.250 | 0.50 | 4.00 | 0.50 |
| Magnesium Stearate (Ligamed MF-2-V) | 4.875 | 0.75 | 6.00 | 0.75 |
| Total Extragranular | 195.000 | 30.00 | 240.00 | 30.00 |
| Core Tablet | 650.000 | 100.00 | 800.00 | 100.00 |

wherein the Core Tablet is optionally coated; preferably coated with coating powder pink
Opadry II 85F250050; more preferably coated with 2-5% (w/w) of coating powder pink
Opadry II 85F250050; more preferably coated with 3% (w/w) of coating powder pink
Opadry II 85F250050. The w/w ratio refers to the content by weight of coating powder
pink Opadry II 85F250050 in respect of the content by weight of the Core Tablet.

The present invention relates to pharmaceutical compositions having the following compositions:

| | Spray Dried Powder: Compound A1 Polymer Ratio | | | | | |
|---|---|---|---|---|---|---|
| | 1/2 | | 1/1 | | 2/1 | |
| | mg/tablet | % w/w | mg/tablet | % w/w | mg/tablet | % w/w |
| Spray Dried Powder | 300.00 | 30.00 | 200.00 | 30.00 | 150.00 | 30.00 |
| comprising Compound A and | | | | | | |
| polymer X | | | | | | |
| Microcrystalline cellulose | 367.50 | 36.75 | 245.00 | 36.75 | 183.75 | 36.75 |
| Croscarmellose Sodium | 25.00 | 2.50 | 16.67 | 2.50 | 12.50 | 2.50 |
| Silica, Colloidal Anhydrous | 5.00 | 0.50 | 3.33 | 0.50 | 2.50 | 0.50 |
| Magnesium Stearate | 2.50 | 0.25 | 1.67 | 0.25 | 1.25 | 0.25 |
| Silicified Microcrystalline | 262.50 | 26.25 | 175.00 | 26.25 | 131.25 | 26.25 |
| cellulose | | | | | | |
| Croscarmellose Sodium | 25.00 | 2.50 | 16.67 | 2.50 | 12.50 | 2.50 |
| Silica, Colloidal Anhydrous | 5.00 | 0.50 | 3.33 | 0.50 | 2.50 | 0.50 |
| Magnesium Stearate | 7.50 | 0.75 | 5.00 | 0.75 | 3.75 | 0.75 |
| Core Tablet | 1000.00 | 100.00 | 666.67 | 100.00 | 500.00 | 100.00 |

wherein polymer X is HPMCAS-LG or HPMC E5; and
wherein the Core Tablet is optionally coated; preferably coated with coating powder pink
Opadry II 85F250050; more preferably coated with 2-5% (w/w) of coating powder pink
Opadry II 85F250050; more preferably coated with 3% (w/w) of coating powder pink
Opadry II 85F250050. The w/w ratio refers to the content by weight of coating powder
pink Opadry II 85F250050 in respect of the content by weight of the Core Tablet.

The present invention relates to pharmaceutical compositions having the following compositions:

| | 65 mg | | 80 mg | |
|---|---|---|---|---|
| | mg/tablet | % w/w | mg/tablet | % w/w |
| Spray Dried Dispersion | 195.000 | 30.00 | 240.00 | 30.00 |
| Powder comprising a | | | | |
| 33.3/66.7 ratio of | | | | |
| Compound A/ | | | | |
| HPMCAS-LG | | | | |
| Microcrystalline cellulose | 238.875 | 36.75 | 294.00 | 36.75 |
| Croscarmellose Sodium | 16.250 | 2.50 | 20.00 | 2.50 |
| Silica, Colloidal Anhydrous | 3.250 | 0.50 | 4.00 | 0.50 |
| Magnesium Stearate | 1.625 | 0.25 | 2.00 | 0.25 |
| Total Intragranular | 455.000 | 70.00 | 560.00 | 70.00 |
| Silicified Microcrystalline | 170.625 | 26.25 | 210.00 | 26.25 |
| cellulose | | | | |
| Croscarmellose Sodium | 16.250 | 2.50 | 20.00 | 2.50 |
| Silica, Colloidal Anhydrous | 3.250 | 0.50 | 4.00 | 0.50 |
| Magnesium Stearate | 4.875 | 0.75 | 6.00 | 0.75 |
| Total Extragranular | 195.000 | 30.00 | 240.00 | 30.00 |
| Core Tablet | 650.000 | 100.00 | 800.00 | 100.00 |

wherein the Core Tablet is optionally coated; preferably coated with coating powder pink
Opadry II 85F250050; more preferably coated with 2-5% (w/w) of coating powder pink
Opadry II 85F250050; more preferably coated with 3% (w/w) of coating powder pink
Opadry II 85F250050. The w/w ratio refers to the content by weight of coating powder
pink Opadry II 85F250050 in respect of the content by weight of the Core Tablet.

The present invention relates to pharmaceutical compositions having the following compositions:

Spray Dried Powder comprising Compound A
and polymer X in a weight-by-weight ratio as
described in any of the other embodiments
Microcrystalline cellulose
Croscarmellose Sodium
Silica, Colloidal Anhydrous
Magnesium Stearate
Silicified Microcrystalline cellulose
Croscarmellose Sodium
Silica, Colloidal Anhydrous
Magnesium Stearate

wherein polymer X is HPMCAS-LG or HPMC E5; and
wherein the Core Tablet is optionally coated; preferably coated with coating powder pink
Opadry II 85F250050; more preferably coated with 2-5% (w/w) of coating powder pink
Opadry II 85F250050; more preferably coated with 3% (w/w) of coating powder pink
Opadry II 85F250050. The w/w ratio refers to the content by weight of coating powder
pink Opadry II 85F250050 in respect of the content by weight of the Core Tablet.

The present invention relates to a process for preparing the amorphous solid dispersion as described herein, comprising the steps of:

a) blending compound A, or a pharmaceutically acceptable salt form thereof; with an orally pharmaceutically acceptable polymer;
b) extruding said blend at a temperature in the range of 20-300° C.

The process for preparing the amorphous solid dispersion as described herein may further comprise preparing particles, wherein said process may further comprise the steps of:

c) grinding the extrudate, and
d) optionally sieving the particles.

The present invention relates to a process for preparing the amorphous solid dispersion as described herein, comprising the steps of:

a) blending compound A, or a pharmaceutically acceptable salt form thereof; with an orally pharmaceutically acceptable polymer and a suitable solvent;
b) spray-drying said blend.

In the process for preparing the amorphous solid dispersion by spray-drying as described herein, the suitable solvent may be selected from: alcohols selected from methanol, ethanol, n-propanol, iso-propanol, and butanol; ketones selected from acetone, methyl ethyl ketone, and methyl iso-butyl ketone; esters selected from ethyl acetate, and propylacetate; acetonitrile; dichloromethane; toluene; 1,1,1-trichloroethane; dimethyl acetamide; dimethylsulfoxide; combinations thereof; a mixture of methanol and dichloromethane, 60:40 (w:w) or 50:50 (w:w); and a mixture of acetone and water 80:20 (w:w).

The present invention relates to a process for preparing the particle comprising compound A in amorphous form or non-crystalline phase, or a pharmaceutically acceptable salt form thereof, said process comprising the step of spray-drying a mixture of compound A, or a pharmaceutically acceptable salt form thereof; and a suitable solvent.

In the process for preparing the particle as described herein before, the process may further comprise the step of spray-drying a mixture of compound A, or a pharmaceutically acceptable salt form thereof; and a suitable solvent; onto the surface of a pharmaceutically acceptable bead.

In the process described herein before, the suitable solvent may be selected from the list defined herein above.

The present invention relates to any one of the processes described herein, further comprising preparing tablets or capsules; said process further comprising blending a therapeutically effective amount of the material obtained from any one of the processes described herein, with pharmaceutically acceptable excipients; and compressing said blend into tablets or filling said blend into capsules.

The present invention relates to an amorphous solid dispersion as described herein, wherein said amorphous solid dispersion is obtainable by melt-extruding a mixture comprising compound A, or a pharmaceutically acceptable salt form thereof; and an orally pharmaceutically acceptable polymer.

The present invention relates to any one of the particles as described herein, wherein said particles are obtainable by grinding the amorphous solid dispersions as described herein, and optionally sieving the obtained particles.

The present invention relates to any one of the amorphous solid dispersions described herein, or any one of the particles described herein, wherein said amorphous solid dispersions or particles are obtainable by spray-drying a mixture comprising compound A, or a pharmaceutically acceptable salt form thereof; an orally pharmaceutically acceptable polymer; and a suitable solvent.

The present invention relates to Compound A in amorphous form or non-crystalline phase, or a pharmaceutically acceptable salt form thereof, or any one of the particles as described herein, wherein said compound A or particles are obtainable by spray-drying a mixture comprising compound A, or a pharmaceutically acceptable salt form thereof, and a suitable solvent.

For the compound A or the particles as obtainable by spray-drying as described herein before, the mixture may be sprayed-dried onto the surface of pharmaceutically acceptable beads.

The present invention relates to Compound A in amorphous form or non-crystalline phase, or a pharmaceutically acceptable salt form thereof, any one of the amorphous solid dispersions as described herein; any one of the particles comprising the amorphous solid dispersion as described herein; or any one of the particles comprising compound A in amorphous form or non-crystalline phase, or a pharmaceutically acceptable salt form thereof, for use in the treatment of a disease, syndrome, condition, or disorder in a subject in need thereof, wherein said disease, syndrome, condition, or disorder is affected by the inhibition of MALT1.

The present invention relates to a method of treating a disease, syndrome, condition, or disorder, wherein said disease, syndrome, condition, or disorder is affected by the inhibition of MALT1, comprising administering to a subject in need thereof a therapeutically effective amount of: (i) compound A in amorphous form or non-crystalline phase, or a pharmaceutically acceptable salt form thereof; (ii) any one of the amorphous solid dispersions as described herein; (iii) any one of the particles comprising the amorphous solid dispersion as described herein; or (iv) any one of the particles comprising compound A in amorphous form or non-crystalline phase, or a pharmaceutically acceptable salt form thereof.

The present invention relates to the use of (i) compound A in amorphous form or non-crystalline phase, or a pharmaceutically acceptable salt form thereof; (ii) any one of the amorphous solid dispersions as described herein; (iii) any one of the particles comprising the amorphous solid dispersion as described herein; or (iv) any one of the particles comprising compound A in amorphous form or non-crystalline phase, or a pharmaceutically acceptable salt form thereof; in the manufacture of a medicament for the treatment of a disease, syndrome, condition, or disorder affected by the inhibition of MALT1.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1: Powder X-Ray Diffraction (PXRD) overlays of amorphous sprayed-dried dispersions (SDDs) of compound A/HPMCAS in ratios 1:2, 1:1, and 2:1 (lot no. BREC-2326-004A, BREC-2326-004B, BREC-2326-004C, respectively), compared to bulk crystalline compound A monohydrate Form III, and crystalline compound A hydrate Form I.

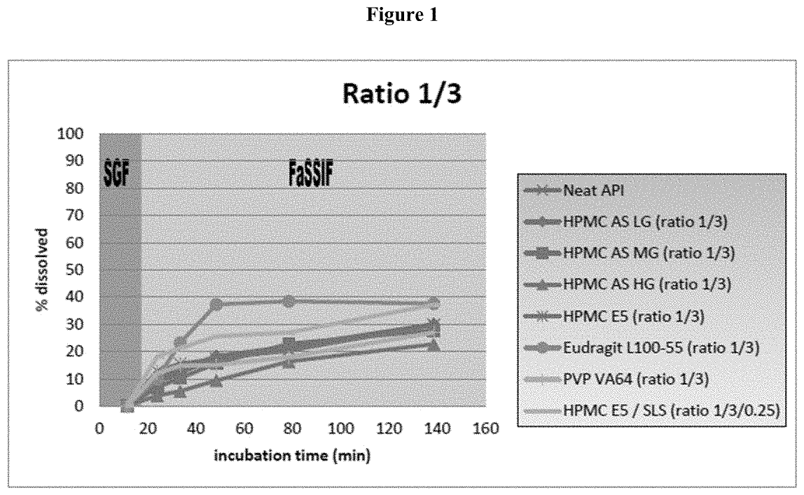

FIG. 2: PXRD overlays of compound A/HPMC E5 amorphous SDDs in ratios 1:2, 1:1, and 2:1 (lot no. BREC-2326-006A, BREC-2326-006B, BREC-2326-006C, respectively), compared to bulk crystalline compound A monohydrate Form III, and crystalline compound A hydrate Form I.

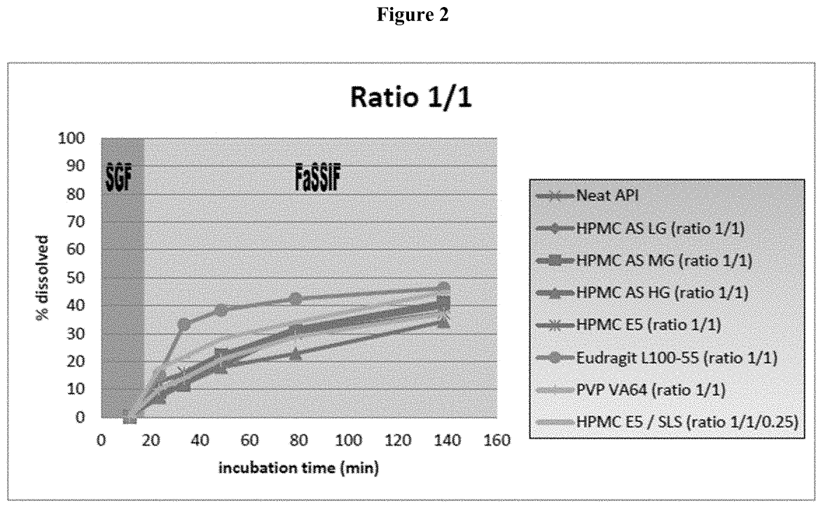

FIG. 3: Scanning electron microscopy (SEM) images at 500× (Top), 1500× (Bottom), Magnification of 33.3% (Left), 50% (Middle), and 66.7% (Right) of compound A/HPMCAS amorphous SDDs. Amorphous SDDs show typical spray dried particle morphology consisting of collapsed spheres with no sign of particle fusing or surface crystallization. In FIG. 3, “MALT-1” refers to Compound A.

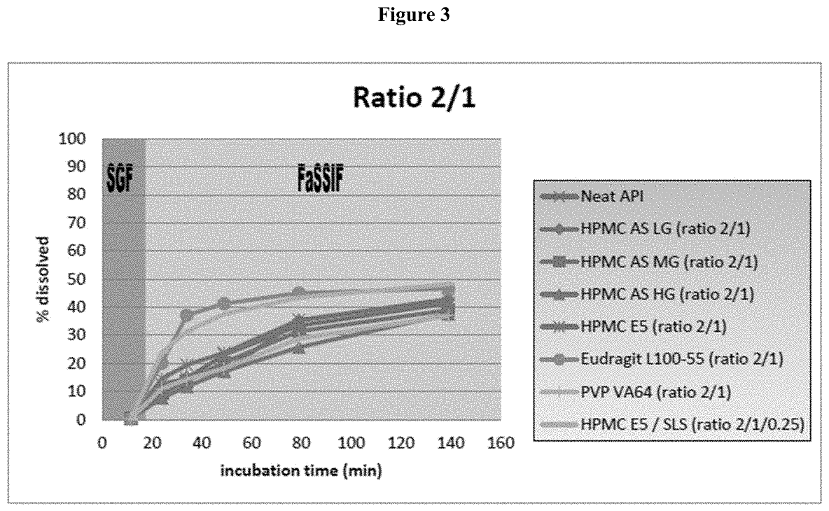

FIG. 4: SEM images at 500× (Top) 1500× (Bottom), Magnification of 33.3% (Left), 50% (Middle), and 66.7% (Right) of compound A/HPMC E5 amorphous SDDs. Amorphous SDDs show typical spray dried particle morphology consisting of collapsed spheres with no sign of particle fusing or surface crystallization. In FIG. 4, “MALT-1” refers to Compound A.

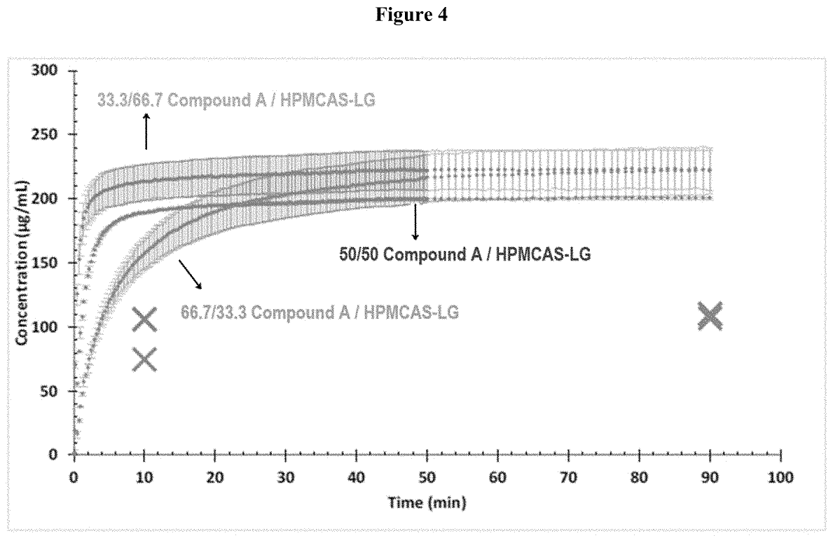

FIG. 5: Solubility in Biorelevant Media for the 33.3/66.7 Compound A/HPMCAS amorphous SDD, lot no. BREC-2326-004A.

FIG. 6: 2-phase dissolution profiles in simulated gastric fluid (SGF) and in fasted state simulated intestinal fluid (FaSSIF) of amorphous solid dispersions, each containing 200 μg of Compound A (Compound A/polymer ratios 1/3(/0.25)).

FIG. 7: 2-phase dissolution profiles (SGF-FaSSIF) of amorphous solid dispersions, each containing 200 μg of Compound A (Compound A/polymer ratios 1/1(/0.25)).

FIG. 8: 2-phase dissolution profiles (SGF-FaSSIF) of amorphous solid dispersions, each containing 200 μg of Compound A (Compound A/polymer ratios 2/1(/0.25)).

FIG. 9: Pion UV-Probe Non-sink Dissolution Test Results for Compound A/HPMCAS amorphous SDDs in pH 6.5 PBS Media (without micelles) with ultracentrifuge samples (X's on Plot, and Tabulated Values).

FIG. 10: Pion UV-Probe Non-sink Dissolution Test Results for Compound A/HPMC-E5 amorphous SDDs in pH 6.5 PBS Media (without micelles) with ultracentrifuge samples (X's on Plot, and Tabulated Values).

FIG. 11: Powder bulk and tapped density results for the prototype compound A amorphous SDD formulations.

FIG. 12: 2-phase dissolution profiles (FaSSIF pH 6.5) of 4 times 100-mg tablets comprising an amorphous solid dispersion of compound A and HPMCAS in ratios 1:1, 1:2, and 2:1.

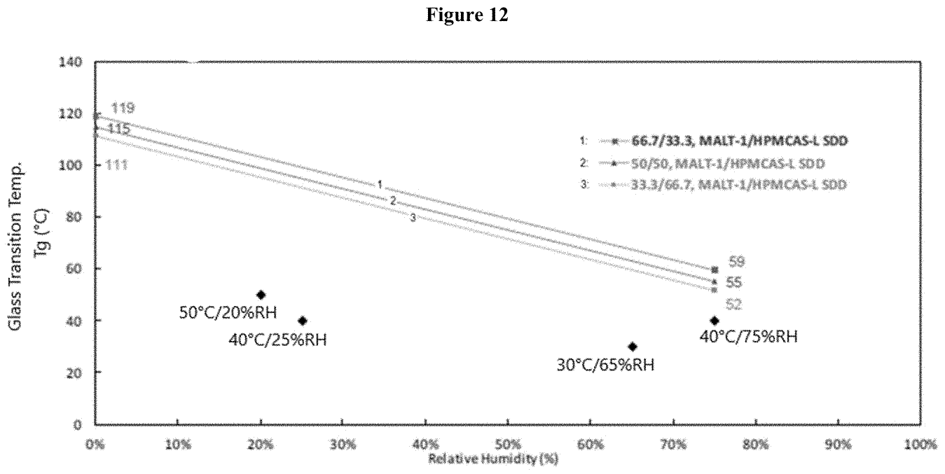

FIG. 13: 2-phase dissolution profiles in fed state simulated intestinal fluid (FeSSIF pH 5.5) of 4 times 100-mg tablets comprising an amorphous solid dispersion of compound A and HPMCAS in ratios 1:1, and 1:2.

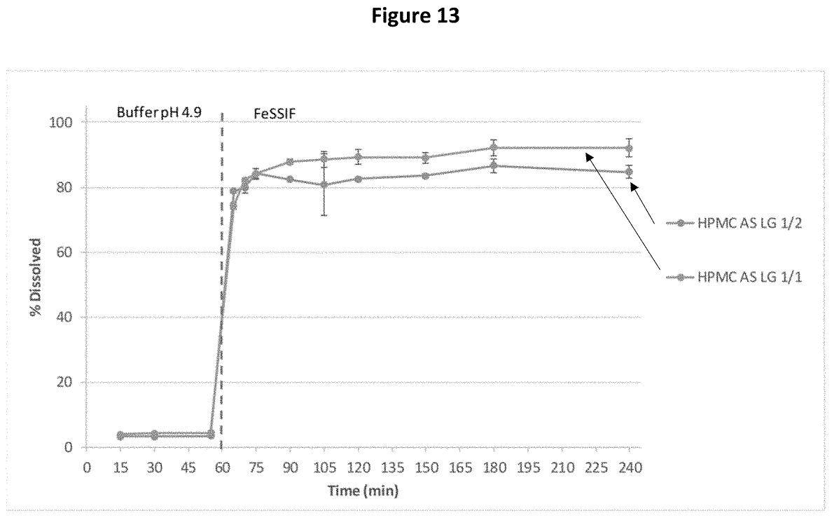

FIG. 14: 2-phase dissolution profiles (FaSSIF pH 6.5) of 4 times 100-mg tablets comprising an amorphous solid dispersion of compound A and HPMC E5 in ratios 1:1, 1:2, and 2:1.

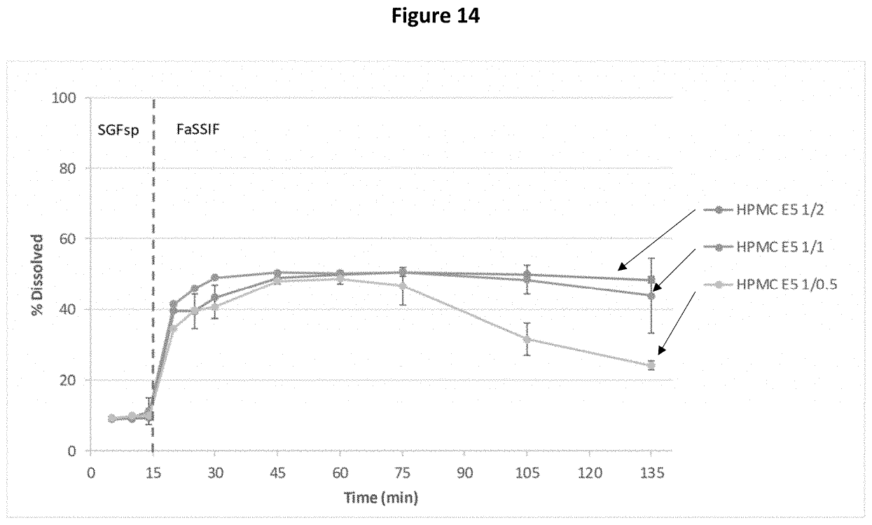

FIG. 15: 2-phase dissolution profiles (FeSSIF pH 5.5) of 4 times 100-mg tablets comprising an amorphous solid dispersion of compound A and HPMC E5 in ratios 1:1, and 1:2.

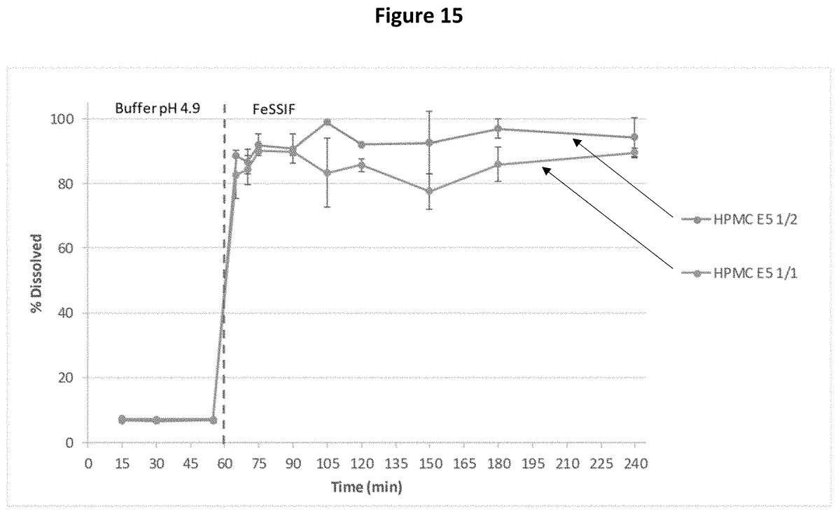

FIG. 16: Area under the plasma concentration-time curve over the last 24-hr dosing interval; (AUCo-24h) in dogs administered normalized doses of: (i) blends with ASDs of compound A and HPMCAS in ratios 1:2 and 2:1; (ii) tablets with ASDs of compound A and HPMCAS in ratios 1:2, 1:1, and 2:1; (iii) tablets with ASDs of compound A and HPMC E5 in ratios 1:2, 1:1, and 2:1; and (iv) blend with ASD of compound A and HPMC E5 in ratio 2:1.

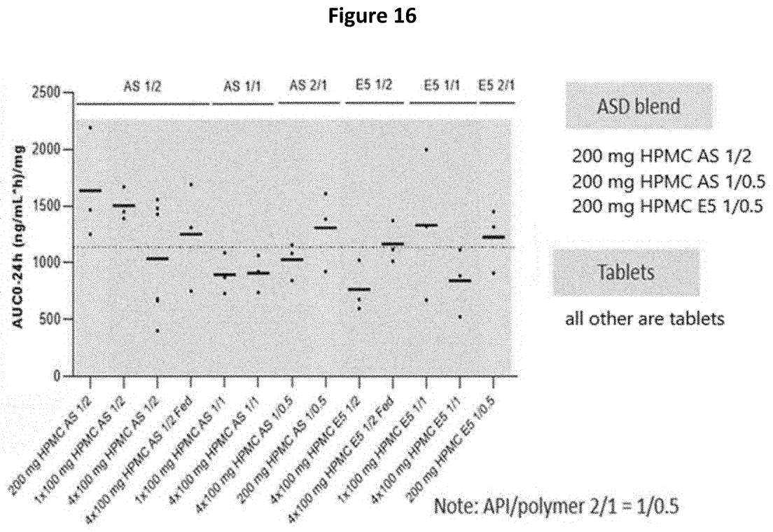

FIG. 17: PXRD pattern of isolated amorphous compound A.

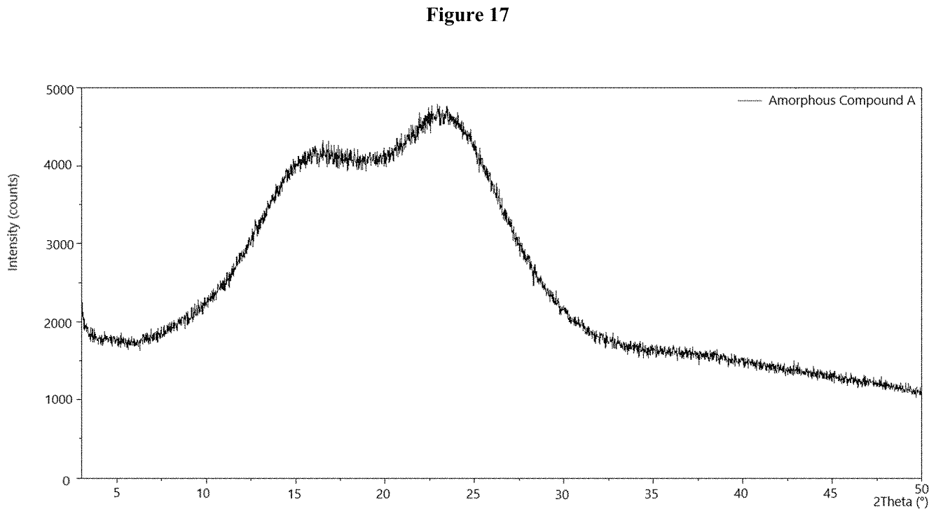

FIG. 18: DSC glass transition temperature (Tg) vs % RH for compound A/HPMCAS amorphous SDDs compared to the International Committee on Harmonization (ICH) stability storage conditions (black diamonds). In FIG. 18, “MALT-1” refers to Compound A.

FIG. 19: DSC Tg vs % RH for compound A/HPMC E5 amorphous SDDs compared to ICH stability storage conditions (black diamonds). In FIG. 19, “MALT-1” refers to Compound A.

FIG. 20: Dissolution rates of different compositions in 900 mL FaSSIF medium at pH 6.5. The different compositions are: 25 mg ASD capsule HPMC AS MG 1/3 in blend; 50 mg ASD capsule HPMC AS MG 1/1 in blend; 50 mg ASD capsule EL100-55 1/1 in blend; 50 mg LFHG PEG1500 (liquid filled) capsule; 100 mg capsule amorphous API in blend; 25 mg ASD capsule EL100-55 1/3 in blend; and 100 mg capsule crystalline API in blend.

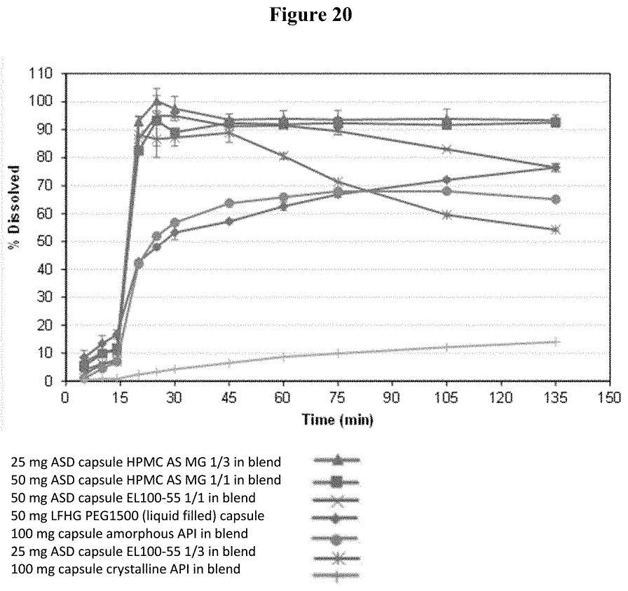

FIG. 21: Preliminary PK results in healthy participants. The different treatments are:

Treatment A (reference capsule): 100 mg Compound A supplied as 2×50 mg LFHG PEG1500 capsules (as described in WO2020/169738); fasted (N=10);
Treatment B (test): 100 mg Compound A supplied as one 100 mg uncoated ASD tablet (Compound A/HPMCAS-LG ratio 1/2 as described in Example 15); fasted (N=10);
Treatment C (test): 100 mg Compound A supplied as one 100 mg uncoated ASD tablet (Compound A/HPMCAS-LG ratio 1/1 as described in Example 15); fasted (N=10);
Treatment D (test): 100 mg Compound A supplied as one 100 mg LFHG PEG1500 capsule (as described in WO2020/169738); fasted (N=10).
LFHG means liquid filled hard gelatin capsules

### DETAILED DESCRIPTION OF THE INVENTION

The present inventions may be understood more readily by reference to the following detailed description, taken in connection with the accompanying examples, which form part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

### Definitions

As employed above and throughout the disclosure, the following terms and abbreviations, unless otherwise indicated, shall be understood to have the following meanings.

In the present disclosure the singular forms “a,”, “an,” and “the” include the plural reference, and reference to a given numerical value includes at least that value, unless the context clearly indicates otherwise. Thus, for example, a reference to “an ingredient” is a reference to one or more of such ingredients and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

Some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions and acceptable error margins, for such given value.

The term “amorphous” refers to solids in which there is no long-range ordering of the molecules. The term amorphous also refers to solids comprising regions of crystallinity and regions that are amorphous. The term amorphous also encompasses semi-crystalline solids.

As used herein, and unless otherwise defined, the terms “treat,” “treating” and “treatment” include the eradication, removal, modification, management or control of a disease, syndrome, condition, or disorder affected by the inhibition of MALT1.

As used herein, and unless otherwise defined, the phrase “therapeutically effective amount” means an amount of compound A effective for treating a disease, syndrome, condition, or disorder affected by the inhibition of MALT1. In one embodiment, the term “therapeutically effective amount” refers to the amount of Compound A, a tautomer, an N-oxide, or a pharmaceutically acceptable salt thereof, that when administered to a subject, is effective to (1) at least partially alleviate, inhibit, prevent, and/or ameliorate a condition, or a disorder or a disease (i) mediated by MALT1; or (ii) associated with MALT1 activity; or (iii) characterized by activity (normal or abnormal) of MALT1; or (2) reduce or inhibit the activity of MALT1; or (3) reduce or inhibit the expression of MALT1; or (4) modify the protein levels of MALT1.

Where doses of the present invention are expressed in relation to the weight of the subject, “mg/kg” is used to specify milligrams of the compound for each kilogram of the subject's body weight.

As used herein, and unless otherwise defined, the phrase “safe therapeutic” means an amount of the therapeutic agent that is safe for treating a disease, syndrome, condition, or disorder affected by the inhibition of MALT1.

The term “pharmaceutically acceptable” means that which is generally safe, non-toxic and neither biologically nor otherwise undesirable and includes that which are approved or approvable for human pharmaceutical use as well as veterinary use, by a regulatory agency of the Federal or a state government or the corresponding agency in countries other than the United States, or that is listed in the U.S. Pharmacopoeia or other generally recognized pharmacopoeia for use in animals, and more particularly, in humans.

The terms “formulation” and “composition” may be used interchangeably in the present disclosure. Whilst composition is usually understood as a broader term of at least combining two or more components, and formulation implies putting together components in appropriate relationships or structures, these terms for the purpose of this disclosure can be used interchangeably.

The terms “excipient” and “carrier” are used interchangeably in the present disclosure. The European Pharmacopoeia (Ph. Eur.) defines an excipient as “any component, other than the active substance(s), present in a medicinal product or used in the manufacture of the product. The intended function of an excipient is to act as the carrier (vehicle or basis) or as a component of the carrier of the active substance(s) and, in so doing, to contribute to product attributes such as stability, biopharmaceutical profile, appearance and patient acceptability and to the ease with which the product can be manufactured. Usually, more than one excipient is used in the formulation of a medicinal product.” The terms vehicle and basis are further defined in the same pharmacopoeia: “A vehicle is the carrier, composed of one or more excipients, for the active substance(s) in a liquid preparation” and “A basis is the carrier, composed of one or more excipients, for the active substance(s) in semi-solid and solid preparations.”

The term “subject” refers to an animal, preferably a mammal, most preferably a human, who has been the object of treatment, observation or experiment.

Throughout the present disclosure, the term “compound A” is meant also to include any pharmaceutically acceptable salt form thereof.

Compound A may be present in other tautomeric arrangements. For example, it is understood that compound A

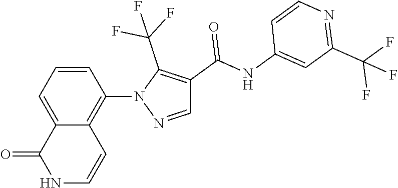 [SMILES: O=C(Nc1ccnc(C(F)(F)F)c1)c1cnn(-c2cccc3c(=O)[nH]ccc23)c1C(F)(F)F]

can occur in another tautomeric arrangement like

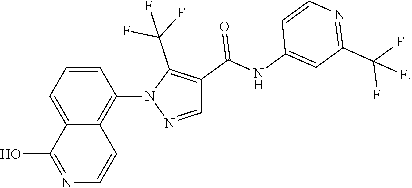 [SMILES: O=C(Nc1ccnc(C(F)(F)F)c1)c1cnn(-c2cccc3c(O)nccc23)c1C(F)(F)F]

Within the context of this invention, Compound A may be in either one of the above tautomeric arrangements or may be a mixture thereof, the exact tautomeric arrangement being unknown. For simplicity, only one possible tautomeric arrangement of the groups of Compound A is utilized in describing the compounds, but it should be clear to a person of ordinary skill in the art that Compound A may be in one of the above tautomeric arrangements or may be a mixture thereof.

Compound A may be converted to the corresponding N-oxide forms following art-known procedures for converting a trivalent nitrogen into its N-oxide form. Said N-oxidation reaction may generally be carried out by reacting the starting material of formula A with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tert-butyl hydroperoxide. Suitable solvents are, for example, water, lower alcohols, e.g. ethanol and the like, hydrocarbons, e.g. toluene, ketones, e.g. 2-butanone, halogenated hydrocarbons, e.g. dichloromethane, and mixtures of such solvents.

The salt forms of the compound A presented herein are typically pharmaceutically acceptable salts, and examples of pharmaceutically acceptable salts are discussed in Berge et al. (1977) “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compound A of the invention, also form part of the invention.

The pharmaceutically acceptable salts include pharmaceutically acceptable acid and base addition salts and are meant to comprise the therapeutically active non-toxic acid and base addition salt forms that the compounds described herein are able to form.

The salts of the present disclosure can be synthesized from the parent compound that contains a basic or acidic moiety by conventional chemical methods such as methods described in “Pharmaceutical Salts: Properties, Selection, and Use”, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, nonaqueous media such as ether, ethyl acetate, ethanol, isopropanol, or acetonitrile are used. The compound of the invention may exist as mono- or di-salts depending upon the pKa of the acid from which the salt is formed.

The pharmaceutically acceptable acid addition salts can conveniently be obtained by treating the base form with such appropriate inorganic acid (such as hydrochloric acid, hydrobromic acid, sulfuric acid, nitric acid, phosphoric acid and the like) or organic acids such (as acetic acid, methanesulfonic acid, maleic acid, tartaric acid, citric acid and the like) in an anion form. Appropriate anions comprise, for example, acetate, 2,2-dichloroacetate, adipate, alginate, ascorbate (e.g. L ascorbate), L-aspartate, benzenesulfonate, benzoate, 4-acetamidobenzoate, butanoate, bicarbonate, bitartrate, bromide, (+) camphorate, camphor-sulphonate, (+)-(1S)-camphor-10-sulphonate, calcium edetate, camsylate, caprate, caproate, caprylate, carbonate, chloride, cinnamate, citrate, cyclamate, dihydrochloride, dodecylsulphate, edetate, estolate, esylate, ethane-1,2-disulphonate, ethanesulphonate, formate, fumarate, galactarate, gentisate, glucoheptonate, gluceptate, gluconate, D-gluconate, glucuronate (e.g. D-glucuronate), glutamate (e.g. L-glutamate), α-oxoglutarate, glycolate, glycollylarsanilate, hexylresorcinate, hippurate, hydrabamine, hydrobromide, hydrochloride, hydriodate, 2-hydroxyethane-sulphonate, hydroxynaphthoate, iodide, isethionate, lactate (e.g. (+)-L-lactate, (±)-DL-lactate), lactobionate, malate, (−)-L-malate, maleate, malonate, mandelate, (±)-DL-mandelate, mesylate, methansulfonate, methylbromide, methylnitrate, methylsulfate, mucate, naphthalene-sulphonate (e.g. naphthalene-2 sulphonate), naphthalene-1,5-disulphonate, 1 hydroxy-2-naphthoate, napsylate, nicotinate, nitrate, oleate, orotate, oxalate, palmitate, pamoate (embonate), pantothenate, phosphate/diphosphate, propionate, polygalacturonate, L pyroglutamate, pyruvate, salicylate, 4-amino-salicylate, sebacate, stearate, subacetate, succinate, sulfate, tannate, tartrate, (+)-L-tartrate, teoclate, thiocyanate, toluenesulphonate (e.g. p-toluenesulphonate), tosylate, triethiodide, undecylenate, valeric acids, as well as acylated amino acids and cation exchange resins.

Conversely said salt forms can be converted by treatment with an appropriate base into the free base form.

The compounds of the present disclosure containing an acidic proton may also be converted into their nontoxic metal or amine addition salt forms by treatment with appropriate organic and inorganic bases in a cation form. Appropriate basic salts comprise those formed with organic cations such as arginine, benzathine, benzylamine, butylamine, chloroprocaine, choline, diethanolamine, dicyclohexylamine, diethanolamine, diethylamine, ethanolamine, ethylamine, ethylenediamine, lysine, meglumine, phenylbenzylamine, piperazine, procaine, triethylamine, tromethamine, and the like; those formed with ammonium ion (i.e., NH₄⁺), quaternary ammonium ion N(CH₃)₄⁺, and substituted ammonium ions (e.g., NH₃R⁺, NH₂R₂⁺, NHR₃⁺, NR₄⁺); and those formed with metallic cations such as aluminum, calcium, lithium, magnesium, potassium, sodium, zinc, and the like. Where the compounds described herein contain an amine function, these may form quaternary ammonium salts, for example by reaction with an alkylating agent according to methods well known to the skilled person. Such quaternary ammonium compounds are within the scope of the compounds presented herein.

Conversely said salt forms can be converted by treatment with an appropriate acid into the free form.

In the amorphous solid dispersions or particles or pharmaceutical formulations as described herein compound A is present in base form or as a pharmaceutically acceptable salt form, such as a pharmaceutically acceptable acid addition salt. Preferably, compound A is present in base form.

The amorphous form and amorphous solid dispersions of the present disclosure can be prepared with a starting material being compound A, in solvate form, or a pharmaceutically acceptable salt form thereof. This solvate form may be Compound A monohydrate or a pharmaceutically acceptable salt form thereof. This solvate form may be Compound A hydrate or a pharmaceutically acceptable salt form thereof.

### Isolated Amorphous Compound A, or a Pharmaceutically Acceptable Salt Form Thereof

The present invention discloses an isolated amorphous form of compound A, or a pharmaceutically acceptable salt form thereof. The present invention discloses an isolated compound A in non-crystalline phase, or a pharmaceutically acceptable salt form thereof.

The present invention discloses an isolated stable amorphous form of compound A, or a pharmaceutically acceptable salt form thereof. The present invention discloses an isolated stable compound A in non-crystalline phase, or a pharmaceutically acceptable salt form thereof.

The Compound A of the present invention is isolated and present in amorphous form or non-crystalline phase in a weight percentage in respect of any crystalline form of compound A, of more than 90% w/w, such as 91% w/w, 92% w/w, 93% w/w, 94% w/w, 95% w/w, 96% w/w, 97% w/w, 98% w/w, 99% w/w, 99.5% w/w, and 99.9%; preferably at least 95% w/w. When a particular percentage by weight of the compound is in amorphous form or non-crystalline phase, the remainder of the compound A may be in any crystalline form of compound A.

This isolated amorphous form of Compound A exhibits no crystalline conversion after exposure at 10% RH, at 50° C., for 28 days.

This isolated amorphous form of Compound A exhibits no crystalline conversion after exposure at 75% RH, at 50° C., for 28 days.

This isolated amorphous form of Compound A exhibits no crystalline conversion after exposure at 75% RH, at 40° C. in open condition, for 6 months.

This isolated amorphous form of Compound A exhibits no crystalline conversion after exposure at 75% RH, at 50° C. in closed condition, for 6 months.

In one embodiment, the amorphous form of Compound A, or a pharmaceutically acceptable salt form thereof, is not formulated in the presence of iron oxide.

In one embodiment, the amorphous form of Compound A, or a pharmaceutically acceptable salt form thereof, is not formulated in the presence of magnesium stearate, SLS, or a combination thereof.

### Amorphous Solid Dispersions and Particles of Compound a, or a Pharmaceutically Acceptable Salt Form Thereof

The term “solid dispersion” defines a system in a solid state (as opposed to a liquid or gaseous state) comprising the components of the present compositions, wherein one component is dispersed more or less evenly throughout the other component or components (the components may include additional pharmaceutically acceptable formulating agents, generally known in the art, such as plasticizers, preservatives and the like). When said dispersion of the components is such that the system is chemically and physically uniform or homogenous throughout or consists of one phase as defined in thermodynamics, such a solid dispersion is called a “solid solution”. Solid solutions are preferred physical systems because the components therein are usually readily bioavailable to the organisms to which they are administered. This advantage can probably be explained by the ease with which said solid solutions can form liquid solutions when contacted with a liquid medium such as the gastrointestinal juices. The ease of dissolution may be attributed at least in part to the fact that the energy required for dissolution of the components from a solid solution is less than that required for the dissolution of components from a crystalline or microcrystalline solid phase.

The solid solution may be a continuous solid solution, in which compound A, or a pharmaceutically acceptable salt form thereof, is molecularly dispersed throughout a matrix formed by the orally pharmaceutically acceptable polymer.

The solid solution may be a discontinuous solid solution, in which compound A, or a pharmaceutically acceptable salt form thereof, is molecularly dispersed throughout a matrix formed by the orally pharmaceutically acceptable polymer. This discontinuous solid solution is partially miscible and presents two phases even though compound A is molecularly dispersed.

The solid solution may be a substitutional solid solution, in which compound A, or a pharmaceutically acceptable salt form thereof, is molecularly dispersed throughout a matrix formed by the orally pharmaceutically acceptable polymer. In this substitutional solid solution, the molecular diameter of compound A differs less than 15% from the matrix (orally pharmaceutically acceptable polymer) diameter. In this case compound A and matrix are substitutional. This substitutional solid solution can be continuous or discontinuous. When discontinuous, two phases are present even though compound A is molecularly dispersed.

The solid solution may be an interstitial solid solution, in which compound A, or a pharmaceutically acceptable salt form thereof, is molecularly dispersed throughout a matrix formed by the orally pharmaceutically acceptable polymer. In this interstitial solid solution, the molecular diameter of compound A is less than 59% of the matrix (orally pharmaceutically acceptable polymer) diameter.

The term “solid dispersion” also comprises dispersions which are less homogenous throughout than solid solutions. Such dispersions are not chemically and physically uniform throughout or comprise more than one phase. For example, the term “solid dispersion” also relates to a system having domains or small regions wherein amorphous, microcrystalline or crystalline drug compound, and/or amorphous, microcrystalline or crystalline orally pharmaceutically acceptable polymer, and optionally amorphous, microcrystalline or crystalline surfactant, are dispersed more or less evenly in another phase comprising a solid solution comprising a drug compound, a polymer, and optionally a surfactant. Said domains are regions within the solid dispersion distinctively marked by some physical feature, small in size, and evenly and randomly distributed throughout the solid dispersion.

The weight-by-weight ratio of the compound A—or a pharmaceutically acceptable salt form thereof—and the orally pharmaceutically acceptable polymer may be in the range of 5:1 to 1:5; preferably in the ratios of 5:1, 4:1, 3:1, 2:1, 1:1, 1:2, 1:3, 1:4, and 1:5 (compound A:orally pharmaceutically acceptable polymer). These ratios may also be expressed as percentages, i.e., as fractions of 100, like “50:50” (or 50/50), which is equivalent to “1:1”, or “66.7:33.3”, which is equivalent to “2:1”; or “33.3:66.7”, which is equivalent to or “1:2”.

Given the therapeutically effective amount of compound A (from about 50 mg to about 1000 mg per 1-, 2-, 3-, 4-, or 5-units dosage forms), the lower limit of the ratio compound:polymer is determined by the maximum amount of mixture that can be processed into one dosage form of practical size.

If the compound:polymer ratio is too high, this means the amount of compound A is relatively high compared to the amount of the orally pharmaceutically acceptable polymer, and then there is the risk that compound A will not dissolve sufficiently in the orally pharmaceutically acceptable polymer, and thus the required bioavailability could not be obtained. However, it will be appreciated that the upper limit of 5:1 may be underestimated for particular orally pharmaceutically acceptable polymers. Amorphous solid dispersions wherein the ratio (compound A):(orally pharmaceutically acceptable polymer) is larger than 5:1 are also meant to be comprised within the scope of the present invention.

As described herein, the particles of the present invention may comprise an orally pharmaceutically acceptable polymer in addition to compound A, or a pharmaceutically acceptable salt form thereof. Preferably, the amorphous solid dispersions and particles of the present invention comprise compound A, or a pharmaceutically acceptable salt form thereof, and an orally pharmaceutically acceptable polymer.

The orally pharmaceutically acceptable polymer in the amorphous solid dispersions and particles according to the present invention, is chosen according to the intended production method. The polymer used for spray-drying may be a polymer that has an apparent viscosity, when dissolved at 20° C., of 1 to 5000 mPa·s, more preferably of 1 to 500 mPa·s, and most preferred of 1 to 100 mPa·s. For Hot Melt Extrusion the molten polymer may have an apparent viscosity of 1 to 1,000,000 Pa·s, preferably 100 to 100,000 Pa·s, and most preferred 500 to 10,000 Pa·s. To a person skilled in the art it is well understood that the given viscosities relate to the chosen formulation and the particular production method, i.e., for 3D printing other viscosities may be preferred.

The orally pharmaceutically acceptable polymer in the amorphous solid dispersions and particles according to the present invention, may be a polymer that has an apparent viscosity in an organic solvent, such as the suitable solvent used in a spray-drying method, of 1 to 5000 mPa·s, more preferably of 1 to 500 mPa·s, and most preferred of 1 to 100 mPa·s.

The orally pharmaceutically acceptable polymer can be selected from the group comprising:
alkylcelluloses such as methylcellulose;
hydroxyalkylcelluloses such as hydroxymethylcellulose, hydroxyethylcellulose, hydroxypropylcellulose and hydroxybutylcellulose;
hydroxyalkyl alkylcelluloses such as hydroxyethyl methylcellulose and hydroxypropyl methylcellulose;
carboxyalkylcelluloses such as carboxymethylcellulose;
alkali metal salts of carboxyalkylcelluloses such as sodium carboxymethylcellulose;
carboxyalkylalkylcelluloses such as carboxymethylethylcellulose;
carboxyalkylcellulose esters;
hydroxypropylmethylcellulose phthalate (HPMCP);
chitin derivates such as chitosan;
polysaccharides such as starches, pectines (sodium carboxymethylamylopectine), cyclodextrins or a derivative thereof, carrageenans, galactomannans, tragacanth, agar agar, gummi arabicum, guar gummi and xanthan gummi;
polyacrylic acids, olyacrylates, and the salts thereof;
polymethacrylic acids, polymethacrylates, the salts and esters thereof, methacrylate copolymers;
polyvinylalcohol (PVA), co-polymers of PVA (e.g., Kollicoat® IR), crospovidone (PVP-CL), polvinylpyrrolidone-polyvinylacetate copolymer (PVP-PVA);
polyalkylene oxides such as polyethylene oxide and polypropylene oxide and copolymers of ethylene oxide and propylene oxide;
polymers of ethylene oxide or polyethylene glycols of molecular weights in the range of 1500-20000, particularly with MW of 4000-6000;
polyvinylpyrrolidone (PVP) of MW ranging from 2500 to 3000000;
Gelita® Collagel; or
any combination thereof;
and optionally a surface-active carrier.

Suitable surface-active carriers or self-emulsifying carriers include Gelucire 44/14, Vitamin E R-alpha-tocopheryl polyethylene glycol 100 succinate (TPGS), Polysorbate 80, alkali dodecylsulphate surfactants such as Sodium Lauryl Sulfate (SLS), or Dioctyl sulfosuccinate sodium salt (DOSS, AOT, docusate sodium), bile salts such as cholic acid, deoxycholic acid and lithocholic acid, cholesterol and esters thereof.

In one embodiment, the orally pharmaceutically acceptable polymers are selected from hydroxypropyl methylcellulose HPMC 2910 5 mPa·s, HPMC-AS, HPMC-E5, Eudragit® E, Eudragit® L, any combination thereof, and optionally mixed with SLS.

### Hydroxypropyl Methylcellulose (HPMC)

HPMC contains sufficient hydroxypropyl and methoxy groups to render it water-soluble. HPMC having a methoxy degree of substitution from about 0.8 to about 2.5 and a hydroxypropyl molar substitution from about 0.05 to about 3.0 are generally water-soluble. Methoxy degree of substitution refers to the average number of methyl ether groups present per anhydroglucose unit of the cellulose molecule. Hydroxy-propyl molar substitution refers to the average number of moles of propylene oxide which have reacted with each anhydroglucose unit of the cellulose molecule. Hydroxypropyl methylcellulose is the United States Adopted Name for hypromellose (see Martindale, The Extra Pharmacopoeia, 29th edition, page 1435). In the four-digit number “2910”, the first two digits represent the approximate percentage of methoxyl groups and the third and fourth digits the approximate percentage composition of hydroxypropoxyl groups. The molecular weight of a water-soluble cellulose ether is generally expressed in terms of the apparent viscosity at 20° C. of an aqueous solution containing two percent by weight of said polymer; e.g., 5 mPa·s is a value indicative of the apparent viscosity of a 2% aqueous solution of HPMC at 20° C.

The molecular weight of the HPMC normally affects both the release profile of the amorphous solid dispersion, as well as its physical properties. A desired release profile can thus be designed by choosing an HPMC of an appropriate molecular weight; for immediate release of the active ingredient from the particles, a low molecular weight polymer is preferred. High molecular weight HPMC is more likely to yield a sustained release pharmaceutical dosage form. Suitable HPMCs include those having a viscosity from about 1 to about 100 mPa·s, in particular form about 3 to about 15 mPa·s, preferably about 5 mPa·s. A preferred type of HPMC having a viscosity of 5 mPa·s., is the commercially available HPMC 2910 5 mPa·s, also known as HPMC E5.

In the case of (compound A):(HPMC E5), the weight-by-weight ratio of the compound A and the orally pharmaceutically acceptable polymer preferably ranges from about 2:1 to about 1:3, or 1:1, or 1:2. The lower limit is determined by practical considerations.

The amorphous solid dispersion may comprise or consist of compound A and HPMC E5. The weight-by-weight ratio of compound A:HPMC E5 in the amorphous solid dispersion as described herein may be in the range from 2:1 to 1:10, preferably from 2:1 to 1:5, more preferably from 2:1 to 1:3 or from 2:1 to 1:2, or 1:1.

An aspect of the invention is a particle comprising or consisting of an amorphous solid dispersion comprising compound A and HPMC E5, in particular wherein the weight-by-weight ratio of compound A:HPMC E5 is 2:1, 1:1, 1:2, or 1:3.

In an aspect of the invention, the amorphous solid dispersion as described herein is obtainable, in particular is obtained, by melt-extruding a mixture comprising compound A and HPMC E5 and optionally subsequently milling said melt-extruded mixture. In an aspect, the particles as described herein are obtainable, in particular are obtained, by melt-extruding a mixture consisting of compound A and HPMC E5 and subsequently milling said melt-extruded mixture. In an aspect, the weight-by-weight ratio of compound A:HPMC E5 is 2:1, 1:1, 1:2, or 1:3.

In an aspect of the invention, the amorphous solid dispersion as described herein is obtainable, in particular is obtained, by spray drying a mixture comprising compound A and HPMC E5 in a suitable solvent. In an aspect, the particles as described herein are obtainable, in particular are obtained, by spray drying a mixture consisting of compound A and HPMC E5 in a suitable solvent. In an aspect, the weight-by-weight ratio of compound A:HPMC E5 is 2:1, 1:1, 1:2, or 1:3.

The amorphous solid dispersion may also comprise or consist of compound A, HPMC E5, and a surface-active carrier, preferably SLS. The weight-by-weight ratio of compound A:HPMC E5:surface-active carrier in the amorphous solid dispersion as described herein may be 1:3:0.25, 1:1:0.25, or 2:1:0.25. The weight-by-weight ratio of compound A:HPMC E5:SLS in the amorphous solid dispersion as described herein may be 1:3:0.25, 1:1:0.25, or 2:1. 0.25.

An aspect of the invention is a particle comprising or consisting of an amorphous solid dispersion comprising compound A, HPMC E5, and a surface-active carrier, in particular wherein the weight-by-weight ratio of compound A:HPMC E5: a surface-active carrier is 1:3:0.25, 1:1:0.25, or 2:1:0.25.

An aspect of the invention is a particle comprising or consisting of an amorphous solid dispersion comprising compound A, HPMC E5, and SLS, in particular wherein the weight-by-weight ratio of compound A:HPMC E5:SLS is 1:3:0.25, 1:1:0.25, or 2:1:0.25.

In an aspect of the invention, the amorphous solid dispersion as described herein is obtainable, in particular is obtained, by melt-extruding a mixture comprising compound A, HPMC E5, and a surface-active carrier, and optionally subsequently milling said melt-extruded mixture. In an aspect, the particles as described herein are obtainable, in particular are obtained, by melt-extruding a mixture consisting of compound A, HPMC E5, and a surface-active carrier, and subsequently milling said melt-extruded mixture. In an aspect, the weight-by-weight ratio of compound A:HPMC E5:surface-active carrier is 1:3:0.25, 1:1:0.25, or 2:1:0.25.

In an aspect of the invention, the amorphous solid dispersion as described herein is obtainable, in particular is obtained, by melt-extruding a mixture comprising compound A, HPMC E5, and SLS, and optionally subsequently milling said melt-extruded mixture. In an aspect, the particles as described herein are obtainable, in particular are obtained, by melt-extruding a mixture consisting of compound A, HPMC E5, and SLS, and subsequently milling said melt-extruded mixture. In an aspect, the weight-by-weight ratio of compound A:HPMC E5:SLS is 1:3:0.25, 1:1:0.25, or 2:1:0.25.

In an aspect of the invention, the amorphous solid dispersion as described herein is obtainable, in particular is obtained, by spray drying a mixture comprising compound A, HPMC E5, and a surface-active carrier, in a suitable solvent. In an aspect, the particles as described herein are obtainable, in particular are obtained, by spray drying a mixture consisting of compound A, HPMC E5, and a surface-active carrier, in a suitable solvent. In an aspect, the weight-by-weight ratio of compound A:HPMC E5:surface-active carrier is 1:3:0.25, 1:1:0.25, or 2:1:0.25.

In an aspect of the invention, the amorphous solid dispersion as described herein is obtainable, in particular is obtained, by spray drying a mixture comprising compound A, HPMC E5, and SLS, in a suitable solvent. In an aspect, the particles as described herein are obtainable, in particular are obtained, by spray drying a mixture consisting of compound A, HPMC E5, and SLS, in a suitable solvent. In an aspect, the weight-by-weight ratio of compound A:HPMC E5:SLS is 1:3:0.25, 1:1:0.25, or 2:1:0.25.

### HPMCAS

HPMCAS or hydroxypropyl methylcellulose acetate succinate or hypromellose acetate succinate is a mixture of acetic acid and monosuccinic acid esters of hydroxypropylmethyl cellulose (IUPAC name:cellulose, 2-hydroxypropyl methyl ether, acetate, hydrogen butanedioate). Different grades are available differentiated based on degree/ratio of substitution (acetyl content, succinoyl content) and particle size (micronized or fine (F), and granular (G)). Because HPMCAS is dissolved in preparing the amorphous solid dispersions of the present invention, the particle size (F or G) is less relevant.

The HPMCAS grades are named differently depending on the manufacturer. For example, Shin-Etsu Chemical Co., Ltd. defines these grades as follows:

| | | | | Mean | Labeled |
|---|---|---|---|---|---|
| Grade | | Acetyl % | Succinoyl % | Particle Size | viscosity |
| Micronized | AS-LF | 8 | 15 | 5 μm | 3 mm²/s |
| | AS-MF | 9 | 11 | | |
| | AS-HF | 12 | 6 | | |
| Granular | AS-LG | 8 | 15 | 1 mm | |
| | AS-MG | 9 | 11 | | |
| | AS-HG | 12 | 6 | | |

Further grades of Shin-Etsu include AQOAT® HPMCAS:HPMCAS-LMP, HPMCAS-MMP, and HPMCAS-HMP, having a medium particle size from about 70 to about 300 μm.

Dow® defines the grades of HPMCAS with the brand Affinisol™ and a code:

AFFINISOL ™ HPMCAS

| | 716 | 912 | 126 |
|---|---|---|---|
| Hydroxypropyl | 5.0-9.0% | 5.0-9.0% | 6.0-10.0% |
| Methoxyl | 20.0-24.0% | 21.0-25.0% | 22.0-26.0% |
| Viscosity* | 2.4-3.6 cP | 2.4-3.6 cP | 2.4-3.6 cP |
| Residue on Ignition | <0.20% | <0.20% | <0.20% |
| Loss on Drying | <5.0% | <5.0% | <5.0% |
| Free Acids | <1.0% | <1.0% | <1.0% |
| Acetate Substitution | 5.0-9.0% | 7.0-11.0% | 10.0-14.0% |
| Succinate Substitution | 14.0-18.0% | 10.0-14.0% | 4.0-8.0% |
| Acetic Acid | 0.5% | 0.5% | 0.5% |

*Viscosity determined as a 2% solution in NaOH solution

Therefore, the HPMCAS, in the amorphous solid dispersions with compound A, may be selected from, and without being limited to, HPMCAS-LG, HPMCAS-MG, HPMCAS-HG, HPMCAS-LF, HPMCAS-MF, HPMCAS-HF, HPMCAS-LMP, HPMCAS-MMP, HPMCAS-HMP, Affinisol™ HPMCAS 716, Affinisol™ HPMCAS 912, and Affinisol™ HPMCAS 126.

The amorphous solid dispersion may comprise or consist of compound A and HPMCAS. The amorphous solid dispersion may comprise or consist of compound A and HPMCAS LG. The amorphous solid dispersion may comprise or consist of compound A and HPMCAS LF. The amorphous solid dispersion may comprise or consist of compound A and HPMCAS MG. The amorphous solid dispersion may comprise or consist of compound A and HPMCAS HG.

The weight-by-weight ratio of compound A:HPMCAS in the amorphous solid dispersion as described herein may be in the range from 2:1 to 1:10, preferably from 2:1 to 1:5, more preferably from 2:1 to 1:3 or from 2:1 to 1:2, or 1:1. The defined weight-by-weight ratios of compound A:HPMCAS are applicable to all the different grades supplied by the manufacturers. The weight-by-weight ratio of compound A:HPMCAS LG may be 2:1, 1:1, 1:2, or 1:3. The weight-by-weight ratio of compound A:HPMCAS LF may be 2:1, 1:1, 1:2, or 1:3. The weight-by-weight ratio of compound A:HPMCAS MG may be 2:1, 1:1, 1:2, or 1:3. The weight-by-weight ratio of compound A:HPMCAS HG may be 2:1, 1:1, 1:2, or 1:3.

An aspect of the invention is a particle comprising or consisting of an amorphous solid dispersion comprising compound A and HPMCAS, in particular wherein the weight-by-weight ratio of compound A:HPMCAS is 2:1, 1:1, 1:2, or 1:3. The HPMCAS in said particle may be selected from any one of the grades available from the manufacturers.

An aspect of the invention is a particle comprising or consisting of an amorphous solid dispersion comprising compound A and HPMCAS LF, in particular wherein the weight-by-weight ratio of compound A:HPMCAS LF is 2:1, 1:1, 1:2, or 1:3.

An aspect of the invention is a particle comprising or consisting of an amorphous solid dispersion comprising compound A and HPMCAS LG, in particular wherein the weight-by-weight ratio of compound A:HPMCAS LG is 2:1, 1:1, 1:2, or 1:3.

An aspect of the invention is a particle comprising or consisting of an amorphous solid dispersion comprising compound A and HPMCAS MG, in particular wherein the weight-by-weight ratio of compound A:HPMCAS MG is 2:1, 1:1, 1:2, or 1:3.

An aspect of the invention is a particle comprising or consisting of an amorphous solid dispersion comprising compound A and HPMCAS HG, in particular wherein the weight-by-weight ratio of compound A:HPMCAS HG is 2:1, 1:1, 1:2, or 1:3.

In an aspect of the invention, the amorphous solid dispersion as described herein is obtainable, in particular is obtained, by melt-extruding a mixture comprising compound A and HPMCAS and optionally subsequently milling said melt-extruded mixture. In an aspect, the particles as described herein are obtainable, in particular are obtained, by melt-extruding a mixture consisting of compound A and HPMCAS and subsequently milling said melt-extruded mixture. In an aspect, the weight-by-weight ratio of compound A:HPMCAS is 2:1, 1:1, 1:2, or 1:3.

In an aspect of the invention, the amorphous solid dispersion as described herein is obtainable, in particular is obtained, by melt-extruding a mixture comprising compound A and HPMCAS LG and optionally subsequently milling said melt-extruded mixture. In an aspect, the particles as described herein are obtainable, in particular are obtained, by melt-extruding a mixture consisting of compound A and HPMCAS LG and subsequently milling said melt-extruded mixture. In an aspect, the weight-by-weight ratio of compound A:HPMCAS LG is 2:1, 1:1, 1:2, or 1:3.

In an aspect of the invention, the amorphous solid dispersion as described herein is obtainable, in particular is obtained, by melt-extruding a mixture comprising compound A and HPMCAS LF and optionally subsequently milling said melt-extruded mixture. In an aspect, the particles as described herein are obtainable, in particular are obtained, by melt-extruding a mixture consisting of compound A and HPMCAS LF and subsequently milling said melt-extruded mixture. In an aspect, the weight-by-weight ratio of compound A:HPMCAS LF is 2:1, 1:1, 1:2, or 1:3.

In an aspect of the invention, the amorphous solid dispersion as described herein is obtainable, in particular is obtained, by melt-extruding a mixture comprising compound A and HPMCAS MG and optionally subsequently milling said melt-extruded mixture. In an aspect, the particles as described herein are obtainable, in particular are obtained, by melt-extruding a mixture consisting of compound A and HPMCAS MG and subsequently milling said melt-extruded mixture. In an aspect, the weight-by-weight ratio of compound A:HPMCAS MG is 2:1, 1:1, 1:2, or 1:3.

In an aspect of the invention, the amorphous solid dispersion as described herein is obtainable, in particular is obtained, by melt-extruding a mixture comprising compound A and HPMCAS HG and optionally subsequently milling said melt-extruded mixture. In an aspect, the particles as described herein are obtainable, in particular are obtained, by melt-extruding a mixture consisting of compound A and HPMCAS HG and subsequently milling said melt-extruded mixture. In an aspect, the weight-by-weight ratio of compound A:HPMCAS HG is 2:1, 1:1, 1:2, or 1:3.

In an aspect of the invention, the amorphous solid dispersion as described herein is obtainable, in particular is obtained, by spray drying a mixture comprising compound A and HPMCAS in a suitable solvent. In an aspect, the particles as described herein are obtainable, in particular are obtained, by spray drying a mixture consisting of compound A and HPMCAS in a suitable solvent. In an aspect, the weight-by-weight ratio of compound A:HPMCAS is 2:1, 1:1, 1:2, or 1:3.

In an aspect of the invention, the amorphous solid dispersion as described herein is obtainable, in particular is obtained, by spray drying a mixture comprising compound A and HPMCAS LG in a suitable solvent. In an aspect, the particles as described herein are obtainable, in particular are obtained, by spray drying a mixture consisting of compound A and HPMCAS LG in a suitable solvent. In an aspect, the weight-by-weight ratio of compound A:HPMCAS LG is 2:1, 1:1, 1:2, or 1:3.

In an aspect of the invention, the amorphous solid dispersion as described herein is obtainable, in particular is obtained, by spray drying a mixture comprising compound A and HPMCAS LF in a suitable solvent. In an aspect, the particles as described herein are obtainable, in particular are obtained, by spray drying a mixture consisting of compound A and HPMCAS LF in a suitable solvent. In an aspect, the weight-by-weight ratio of compound A:HPMCAS LF is 2:1, 1:1, 1:2, or 1:3.

In an aspect of the invention, the amorphous solid dispersion as described herein is obtainable, in particular is obtained, by spray drying a mixture comprising compound A and HPMCAS MG in a suitable solvent. In an aspect, the particles as described herein are obtainable, in particular are obtained, by spray drying a mixture consisting of compound A and HPMCAS MG in a suitable solvent. In an aspect, the weight-by-weight ratio of compound A:HPMCAS MG is 2:1, 1:1, 1:2, or 1:3.

In an aspect of the invention, the amorphous solid dispersion as described herein is obtainable, in particular is obtained, by spray drying a mixture comprising compound A and HPMCAS HG in a suitable solvent. In an aspect, the particles as described herein are obtainable, in particular are obtained, by spray drying a mixture consisting of compound A and HPMCAS HG in a suitable solvent. In an aspect, the weight-by-weight ratio of compound A:HPMCAS HG is 2:1, 1:1, 1:2, or 1:3.

### Eudragit®

Copolymers derived from esters of acrylic and methacrylic acid (poly(meth)acrylates) are known in the industry as Eudragit®. Eudragit® is the brand name for a diverse range of polymethacrylate-based copolymers. Different grades are available. In an aspect of the invention, the Eudragit® in the dispersions with compound A is Eudragit® L 100-55, which contains an anionic copolymer based on methacrylic acid and ethyl acrylate (CAS number 25212-88-8; Chemical/IUPAC name:Poly(methacrylic acid-co-ethyl acrylate) 1:1) (Evonik Industries). In an aspect of the invention, the Eudragit® is Eudragit (Rohm GmbH, Germany), which is an aminoalkyl methacrylate copolymer, more in particular poly(butyl methacrylate, (2-dimethylaminoethyl)methacrylate, methyl methacrylate) (1:2:1). This basic polymethacrylate is soluble in gastric fluid up to pH 5. Eudragit® E 100 is a solvent-free Eudragit® E solid substance. In an aspect of the invention, the Eudragit® in the dispersions with compound A is Eudragit® E 100, which is a cationic copolymer based on dimethylaminoethyl methacrylate, butyl methacrylate, and methyl methacrylate (CAS number 24938-16-7; Chemical/IUPAC name:Poly(butyl methacrylate-co-(2-dimethylaminoethyl) methacrylate-co-methyl methacrylate) 1:2:1 (Evonik Industries).

An aspect of the invention is an amorphous solid dispersion comprising or consisting of compound A and Eudragit® L 100-55. An aspect of the invention is an amorphous solid dispersion comprising or consisting of compound A and Eudragit® E 100. An aspect of the invention is an amorphous solid dispersion comprising or consisting of compound A and a poly(meth)acrylate copolymer.

In an aspect of the invention, the weight-by-weight ratio of compound A:poly(meth)acrylate copolymer in the amorphous solid dispersion as described herein is in the range from 2:1 to 1:10, preferably from 2:1 to 1:5, more preferably from 2:1 to 1:3 or from 2:1 to 1:2, or 1:1. In an aspect of the invention, the weight-by-weight ratio of compound A:Eudragit® L 100-55 ranges from 2:1 to 1:10, preferably from 2:1 to 1:5, more preferably from 2:1 to 1:3 or from 2:1 to 1:2, or 1:1. In an aspect of the invention, the weight-by-weight ratio of compound A:Eudragit® L 100-55 is 1:3. In an aspect of the invention, the weight-by-weight ratio of compound A:Eudragit® L 100-55 is 1:2. In an aspect of the invention, the weight-by-weight ratio of compound A:Eudragit® L 100-55 is 1:1. In an aspect of the invention, the weight-by-weight ratio of compound A:Eudragit® L 100-55 is 2:1.

An aspect of the invention is a particle comprising or consisting of an amorphous solid dispersion comprising compound A and a poly(meth)acrylate copolymer, in particular wherein the weight-by-weight ratio of compound A:poly(meth)acrylate copolymer is 1:3, 1:2, 1:1, or 2:1.

An aspect of the invention is a particle comprising or consisting of an amorphous solid dispersion comprising compound A and Eudragit® L 100-55, in particular wherein the weight-by-weight ratio of compound A:Eudragit® L 100-55 is 1:3, 1:2, 1:1, or 2:1. An aspect of the invention is a particle comprising or consisting of an amorphous solid dispersion comprising compound A and Eudragit® E 100, in particular wherein the weight-by-weight ratio of compound A:Eudragit® E 100 is 1:3, 1:2, 1:1, or 2:1. An aspect of the invention is a particle comprising or consisting of an amorphous solid dispersion comprising compound A and a poly(meth)acrylate copolymer, in particular wherein the weight-by-weight ratio of compound A:poly(meth)acrylate copolymer is 1:3, 1:2, 1:1, or 2:1.

An aspect of the invention is a particle comprising or consisting of an amorphous solid dispersion consisting of compound A and Eudragit® L 100-55, in particular wherein the weight-by-weight ratio of compound A:Eudragit® L 100-55 is 1:3, 1:2, 1:1, or 2:1. An aspect of the invention is a particle comprising or consisting of an amorphous solid dispersion consisting of compound A and Eudragit® E 100, in particular wherein the weight-by-weight ratio of compound A:Eudragit® E 100 is 1:3, 1:2, 1:1, or 2:1. An aspect of the invention is a particle comprising or consisting of an amorphous solid dispersion consisting of compound A and a poly(meth)acrylate copolymer, in particular wherein the weight-by-weight ratio of compound A:poly(meth)acrylate copolymer is 1:3, 1:2, 1:1, or 2:1.

In an aspect of the invention, the amorphous solid dispersion as described herein is obtainable, in particular is obtained, by melt-extruding a mixture comprising compound A and a poly(meth)acrylate copolymer, and optionally subsequently milling said melt-extruded mixture. In an aspect, the particles as described herein are obtainable, in particular are obtained, by melt-extruding a mixture comprising compound A and a poly(meth)acrylate copolymer and subsequently milling said melt-extruded mixture. In an aspect, the weight-by-weight ratio of compound A:poly(meth)acrylate copolymer is 1:3, 1:2, 1:1, or 2:1.

In an aspect of the invention, the amorphous solid dispersion as described herein is obtainable, in particular is obtained, by melt-extruding a mixture comprising compound A and Eudragit® L 100-55, and optionally subsequently milling said melt-extruded mixture. In an aspect, the particles as described herein are obtainable, in particular are obtained, by melt-extruding a mixture comprising compound A and Eudragit® L 100-55 and subsequently milling said melt-extruded mixture. In an aspect, the weight-by-weight ratio of compound A:Eudragit® L 100-55 is 1:3, 1:2, 1:1, or 2:1.

In an aspect of the invention, the amorphous solid dispersion as described herein is obtainable, in particular is obtained, by melt-extruding a mixture comprising compound A and Eudragit® E 100, and optionally subsequently milling said melt-extruded mixture. In an aspect, the particles as described herein are obtainable, in particular are obtained, by melt-extruding a mixture comprising compound A and Eudragit® E 100 and subsequently milling said melt-extruded mixture. In an aspect, the weight-by-weight ratio of compound A:Eudragit® E 100 is 1:3, 1:2, 1:1, or 2:1.

In an aspect of the invention, the amorphous solid dispersion as described herein is obtainable, in particular is obtained, by spray drying a mixture comprising compound A and a poly(meth)acrylate copolymer in a suitable solvent. In an aspect, the particles as described herein are obtainable, in particular are obtained, by spray drying a mixture comprising compound A and a poly(meth)acrylate copolymer in a suitable solvent. In an aspect, the weight-by-weight ratio of compound A:poly(meth)acrylate copolymer is 1:3, 1:2, 1:1, or 2:1.

In an aspect of the invention, the amorphous solid dispersion as described herein is obtainable, in particular is obtained, by spray drying a mixture comprising compound A and Eudragit® L 100-55 in a suitable solvent. In an aspect, the particles as described herein are obtainable, in particular are obtained, by spray drying a mixture comprising compound A and Eudragit® L 100-55 in a suitable solvent. In an aspect, the weight-by-weight ratio of compound A:Eudragit® L 100-55 is 1:3, 1:2, 1:1, or 2:1.

In an aspect of the invention, the amorphous solid dispersion as described herein is obtainable, in particular is obtained, by spray drying a mixture comprising compound A and Eudragit® E 100 in a suitable solvent. In an aspect, the particles as described herein are obtainable, in particular are obtained, by spray drying a mixture comprising compound A and Eudragit® E 100 in a suitable solvent. In an aspect, the weight-by-weight ratio of compound A:Eudragit® E 100 is 1:3, 1:2, 1:1, or 2:1.

### Polyvinylpyrrolidone (PVP)

The group of polyvinylpyrolidones (PVP) includes crosslinked-polyvinylpyrolidone (crospovidone), and polyvinylpyrolidone vinyl acetate copolymer (PVP-VA64), and may be employed in the amorphous solid dispersions and particles presented herein.

Names and abbreviations for polyvinylpyrrolidone include, but are not limited to, PVP, povidone and crospovidone. Crospovidone is a crosslinked homopolymer of vinyl pyrrolidone.

Names and abbreviations for a copolymer of 1-vinyl-2-pyrrolidone and vinyl acetate in a ratio of 6:4 by mass (PVPVA64) include, but are not limited to, copolyvidone, copovidum, and copovidone. Examples of commercially available PVPVA64 are Kollidon® VA64, Kollidon® VA64 Fine, Luviskol VA64®, and Plasdone S-630®.

The average molecular weight of polyvinylpyrrolidone (PVP) is not critical and any average molecular weight of PVP (see e.g. Handbook of Pharmaceutical Excipients, 3nd Ed (2000), 433-439, American Pharmaceutical Association Washington and The Pharmaceutical Press London), may be used, but preferably PVP ranges from 10,000 to 100,000 (K=17-96), most preferably PVP with K=30, because the capability of preventing crystallization of compound A and the solubility in the solvent are well balanced.

The crospovidone (or cross-polyvinylpyrrolidone, cross-PVP) described in reference of Handbook of Pharmaceutical Excipients, 3nd Ed (2000), 163-164, American Pharmaceutical Association Washington and The Pharmaceutical Press London) may be used.

The average molecular weight of polyvinylpyrrolidone/vinylacetate copolymer (PVP VA64 or PVP-VA or copolyvidone) is also not critical and any average molecular weight of PVP-VA64 can be used, but preferably, PVP-VA64 with K value 24-36 should be used. PVP VA 64 is water-soluble vinylpyrrolidone-vinyl acetate copolymer contains the two components in a ratio of 6:4. Because of its vinyl acetate component, PVP VA 64 is somewhat more hydrophobic, less hygroscopic and has greater elasticity than PVP.

In the case of (compound A):(PVP VA64), the weight-by-weight ratio of the compound A and the orally pharmaceutically acceptable polymer preferably ranges from about 2:1 to about 1:3, or 1:2, or 1:1. The lower limit is determined by practical considerations.

The amorphous solid dispersion may comprise or consist of compound A and PVP VA64. The weight-by-weight ratio of compound A:PVP VA64 in the amorphous solid dispersion as described herein may be in the range from 2:1 to 1:10, preferably from 2:1 to 1:5, more preferably from 2:1 to 1:3 or from 2:1 to 1:2, or 1:1.

An aspect of the invention is a particle comprising or consisting of an amorphous solid dispersion comprising compound A and PVP VA64, in particular wherein the weight-by-weight ratio of compound A:PVP VA64 is 2:1, 1:1, 1:2, or 1:3.

In an aspect of the invention, the amorphous solid dispersion as described herein is obtainable, in particular is obtained, by melt-extruding a mixture comprising compound A and PVP VA64 and optionally subsequently milling said melt-extruded mixture. In an aspect, the particles as described herein are obtainable, in particular are obtained, by melt-extruding a mixture consisting of compound A and PVP VA64 and subsequently milling said melt-extruded mixture. In an aspect, the weight-by-weight ratio of compound A:PVP VA64 is 2:1, 1:1, 1:2, or 1:3.

In an aspect of the invention, the amorphous solid dispersion as described herein is obtainable, in particular is obtained, by spray drying a mixture comprising compound A and PVP VA64 in a suitable solvent. In an aspect, the particles as described herein are obtainable, in particular are obtained, by spray drying a mixture consisting of compound A and PVP VA64 in a suitable solvent. In an aspect, the weight-by-weight ratio of compound A:PVP VA64 is 2:1, 1:1, 1:2, or 1:3.

### Methods of Preparation of Amorphous Solid Dispersions and Particles

The amorphous solid dispersions and particles according to the present invention can be prepared by first preparing an amorphous solid dispersion of the components, and then optionally grinding or milling that dispersion. Various techniques exist for preparing amorphous solid dispersions including melt-extrusion, spray-drying, antisolvent precipitation, solution-evaporation, KinetiSol®, and the like.

The melt-extrusion process comprises the following steps:

a) mixing compound A and an orally pharmaceutically acceptable polymer,
b) optionally blending additives with the thus obtained mixture,
c) heating the thus obtained blend until one obtains a homogenous melt,
d) forcing the thus obtained melt through one or more nozzles; and
e) cooling the melt till it solidifies.

The terms “melt” and “melting” should be interpreted broadly. For our purposes, these terms not only mean the alteration from a solid state to a liquid state, but can also refer to a transition to a glassy state or a rubbery state, and in which it is possible for one component of the mixture to get embedded more or less homogeneously into the other. In particular cases, one component will melt, and the other component(s) will dissolve in the melt thus forming a solution, which upon cooling may form a solid solution having advantageous dissolution properties.

One important parameter of melt extrusion is the temperature at which the melt-extruder is operating. It was found that the operating temperature can easily range between about 20° C. and about 300° C., more preferably between about 70° C. and 250° C., preferably ranges between about 160° C. and about 190° C., more preferably ranges between about 160° C. and 175° C. The lower temperature limit depends on the solubility of compound A in the orally pharmaceutically acceptable polymer and on the viscosity of the mixture. When compound A is not completely dissolved in the orally pharmaceutically acceptable polymer, the extrudate will not have the required bioavailability; when the viscosity of the mixture is too high, the process of melt extrusion will be difficult. A person skilled in the art will easily recognize the most appropriate temperature range to be used.

The throughput rate is also of importance because even at relatively low temperatures the orally pharmaceutically acceptable polymer may start to decompose when it remains too long in contact with the heating element.

It will be appreciated that the person skilled in the art will be able to optimize the parameters of the melt extrusion process within the above given ranges. The working temperatures will also be determined by the kind of extruder or the kind of configuration within the extruder that is used. Most of the energy needed to melt, mix and dissolve the components in the extruder can be provided by the heating elements. However, the friction of the material within the extruder may also provide a substantial amount of energy to the mixture and aid in the formation of a homogenous melt of the components.

A person skilled in the art will easily recognize the most appropriate extruder, such as, for example, a single screw, a twin-screw extruder or a multi-screw extruder, for the preparation of the subject-matter of the present invention. Suitable extruders that may be used are the Haake mini-extruder, Leistritz 18 mm extruder, and the Leistritz 27 mm extruder.

Spray-drying of a solution of the components also yields an amorphous solid dispersion of said components and may be a useful alternative to the melt-extrusion process, particularly in those cases where the orally pharmaceutically acceptable polymer is not sufficiently stable to withstand the extrusion conditions and where residual solvent can effectively be removed from the amorphous solid dispersion. Yet another possible preparation is solution-evaporation, which consists of preparing a solution of the components, pouring said solution onto a large surface so as to form a thin film, and evaporating the solvent therefrom.

Solvents suitable for spray-drying can be any organic solvent in which compound A and the orally pharmaceutically acceptable polymer are miscible. In an aspect of the invention, the boiling point of the solvent is lower than the Tg (glass transition temperature) of the amorphous solid dispersion. In addition, the solvent should have relatively low toxicity and be removed from the dispersion to a level that is acceptable according to The International Committee on Harmonization (ICH) guidelines. Removal of solvent to this level may require a post drying step such as for instance tray-drying, subsequent to the spray-drying process. Solvents include alcohols such as methanol, ethanol, n-propanol, iso-propanol, and butanol, in particular methanol; ketones such as acetone, methyl ethyl ketone and methyl iso-butyl ketone; esters such as ethyl acetate and propylacetate; and various other solvents such as acetonitrile, dichloromethane, toluene, and 1,1,1-trichloroethane. Lower volatility solvents such as dimethyl acetamide or dimethylsulfoxide can also be used. In an aspect of the invention, the solvent suitable for spray drying is a mixture of solvents. In an aspect of the invention the solvent for spray drying is a mixture of an alcohol and dichloromethane, in particular a mixture of methanol and dichloromethane, more in particular a mixture of methanol and dichloromethane 60:40 (w:w) or 50:50 (w:w), 40:60 (w:w) being preferred. In an aspect of the invention the solvent for spray drying is a mixture of acetone and water 80:20 (w:w).

The amorphous solid dispersion product is milled or ground to particles. Alternatively, the particles obtained from the methods described herein—methods to prepare the amorphous form of compound A, with or without the orally pharmaceutically acceptable polymer—have already the desired particle size.

The particles comprising the amorphous solid dispersion, as described herein, have a volume weighted particle size distribution Dv50, as measured by a static light scattering instrument, of from about 20 μm to about 90 μm, preferably from about 25 μm to about 80 μm, more preferably from about 25 μm to about 65 μm. The particles have a Dv10 of volume weighted particle size distribution from about 1 μm to about 15 μm; and the Dv90 of the volume weighted particle size distribution is from about 40 μm to about 200 μm.

Particles obtained by spray drying usually have already the mentioned Dv50, Dv10 and Dv90, mentioned above. Milling or grinding is few times needed after spray-drying, but can also be applied.

As used herein, the terms Dv50, Dv10 and Dv90 have their conventional meaning as known to the person skilled in the art and can be measured by art-known particle size measuring techniques such as, for example, static light scattering, sedimentation field flow fractionation, photon correlation spectroscopy, laser diffraction or disk centrifugation.

By “Dv50” it is meant that 50% of the volume weighted of the particles has a particle size of from about 20 μm to about 90 μm. By “Dv90” it is meant that 90% of the volume weighted of the particles has a particle size of from about 40 μm to about 200 μm. By “Dv10” it is meant that 10% of the volume weighted of the particles has a particle size of from about 1 μm to about 15 μm.

Usually volume and weight distribution result in the same or about the same value for the average particle size.

The particle size proves to be an important factor determining the speed, in particular the flowability, with which a particular dosage form can be manufactured on a large scale of a particular dosage form or formulation, and the quality of the final product. The smaller the particles, the faster the tableting speed can be, without detrimental effects on their quality. Too small particles often cause sticking on the tablet punches and manufacturability issues.

Particles of the dimensions mentioned herein can be obtained by sieving them through nominal standard test sieves as described in the CRC Handbook, 64th ed., page F-114. Nominal standard sieves are characterized by the mesh/hole width (μm), DIN 4188 (mm), ASTM E 11-70 (No), Tyler® (mesh) or BS 410 (mesh) values. Throughout this description, and in the claims hereinafter, particle sizes are designated by reference to the mesh/hole width in μm and to the corresponding Sieve No. in the ASTM E11-70 standard.

Preferred are particles wherein compound A is in a non-crystalline phase as these have an intrinsically faster dissolution rate than those wherein part or all of compound A is in a microcrystalline or crystalline form.

In an embodiment, the amorphous solid dispersion is in the form of a solid solution comprising compound A and an orally pharmaceutically acceptable polymer. Alternatively, it may be in the form of a dispersion wherein i) amorphous and/or microcrystalline compound A, and (ii) an amorphous or microcrystalline orally pharmaceutically acceptable polymer are dispersed more or less evenly in a solid solution comprising (i) and (ii).

The amorphous solid dispersions and particles as described herein may further comprise one or more pharmaceutically acceptable excipients such as, for example, plasticizers, flavors, colorants, preservatives and the like. Said excipients should not be heat-sensitive, in other words, they should not show any appreciable degradation or decomposition at the working temperature of the melt-extruder.

In formulations (compound A:HPMC E5, or compound A:HPMC E5:SLS), the amount of plasticizer may be small, in the order of 0% to 15% (w/w), preferably less than 5% (w/w). With other orally pharmaceutically acceptable polymers though, plasticizers may be employed in much different, often higher amounts, because plasticizers as mentioned hereinbelow lower the temperature at which a melt is formed of compound A, the orally pharmaceutically acceptable polymer, and plasticizer; and this lowering of the melting point is advantageous where the polymer has limited thermal stability.

Suitable plasticizers are pharmaceutically acceptable and include low molecular weight polyalcohols such as ethylene glycol, propylene glycol, 1,2 butylene glycol, 2,3-butylene glycol, styrene glycol; polyethylene glycols such as diethylene glycol, triethylene glycol, tetraethylene glycol; other polyethylene glycols having a molecular weight lower than 1,000 g/mol; polypropylene glycols having a molecular weight lower than 200 g/mol; glycol ethers such as monopropylene glycol monoisopropyl ether; propylene glycol monoethyl ether; diethylene glycol monoethyl ether; ester type plasticizers such as sorbitol lactate, ethyl lactate, butyl lactate, ethyl glycolate, allyl glycollate; and amines such as monoethanolamine, diethanolamine, triethanolamine, monoisopropanolamine; triethylenetetramine, 2-amino-2-methyl-1,3-propanediol and the like. Of these, the low molecular weight polyethylene glycols, ethylene glycol, low molecular weight polypropylene glycols and especially propylene glycol are preferred.

In an aspect of the invention, the particles or amorphous solid dispersions as described herein do not contain a plasticizer.

Once the extrudate or spray-dried material is obtained, it can be milled and sieved, and it can be used as ingredient to make pharmaceutical dosage forms.

In an alternative embodiment, compound A may be mixed with a suitable solvent, without the presence of the orally pharmaceutically acceptable polymer, and sprayed dried. The obtained particle comprising amorphous compound A, or a pharmaceutically acceptable salt thereof, may have a volume weighted particle size distribution Dv50, as measured by a static light scattering instrument, of from about 1 μm to about 100 μm, preferably from about 5 μm to about 80 μm, more preferably from about 25 μm to about 75 μm. The obtained particle comprising amorphous compound A, or a pharmaceutically acceptable salt thereof, may have a Dv10 of volume weighted particle size distribution from about 0.1 μm to about 15 μm; and the Dv90 of the volume weighted particle size distribution is from about 3 μm to about 250 μm.

In an alternative embodiment, compound A may be mixed with a suitable solvent, without the presence of the orally pharmaceutically acceptable polymer, and sprayed dried onto the granular surface of excipients or sugar spheres to produce either granules ready for tableting or drug-coated pellets for encapsulation in one step.

Alternatively, a solution of compound A and an orally pharmaceutically acceptable polymer, in an organic solvent, as described herein above in the spray-drying processes, may be used to coat inert cores or beads. A solid solution of compound A in the orally pharmaceutically acceptable polymer is produced upon coating (cosolvent evaporation) and controlled drying of coated beads in a closed Wurster process. As this thin film dissolves in water or gastrointestinal fluid, the molecularly dispersed compound A is released at supersaturated concentration. The orally pharmaceutically acceptable polymer acts as a stabilizer to inhibit recrystallization of compound A. The supersaturated solutions of compound A are sufficiently stable to allow for absorption and distribution.

In yet another alternative embodiment, a solution of compound A in an organic solvent, without the orally pharmaceutically acceptable polymer, may be used to coat directly inert cores or beads by cosolvent evaporation and controlled drying of coated beads in a closed Wurster process.

These inert cores or beads comprise pharmaceutically acceptable materials, have appropriate dimensions and firmness. Examples of such materials are polymers e.g. plastic resins; inorganic substances, e.g. silica, glass, hydroxyapatite, salts (sodium or potassium chloride, calcium or magnesium carbonate) and the like; organic substances, e.g. activated carbon, acids (citric, fumaric, tartaric, ascorbic and the like acids), and saccharides and derivatives thereof. Particularly suitable materials are saccharides such as sugars, oligosaccharides, polysaccharides and their derivatives, for example, glucose, rhamnose, galactose, lactose, sucrose, mannitol, sorbitol, dextrin, maltodextrin, cellulose, sodium carboxymethyl cellulose, starches (maize, rice, potato, wheat, tapioca) and the like saccharides.

The core may have a diameter of about 250 to about 600 μm (30-60 mesh), of about 250 to about 500 μm (35-60 mesh), of about 250 to about 425 μm (40-60 mesh), of about 250 to about 355 μm (45-60 mesh), or of about 212 to 300 μm (50-70 mesh).

An example of suitable cores are 25-30 mesh sugar spheres (NF XVII, p 1989) which consist of 67.5%-91.5% (w/w) sucrose, the remainder being starch and possibly also dextrines, and which are pharmaceutically inert or neutral.

Pellets, beads or cores of the dimensions mentioned herein can be obtained by sieving through nominal standard test sieves as described in the CRC Handbook, 64th ed., page F-1 14. Nominal standard sieves are characterized by the mesh/hole width (μm), DIN 4188 (mm), ASTM E 11-70 (No), Tyler® (mesh) or BS 410 (mesh) standard values.

### Pharmaceutical Compositions

An aspect of the invention is a pharmaceutical formulation comprising a pharmaceutically acceptable carrier and an amorphous solid dispersion as described herein.

An aspect of the invention is a pharmaceutical formulation comprising a pharmaceutically acceptable carrier and particles as described herein.

The particles of the present invention can be formulated into pharmaceutical dosage forms comprising a therapeutically effective amount of particles comprising an amorphous solid dispersion, said amorphous solid dispersion comprising compound A and an orally pharmaceutically acceptable polymer. Although, at first instance, pharmaceutical dosage forms for oral administration such as tablets and capsules are envisaged, the particles of the present invention can also be used to prepare pharmaceutical dosage forms e.g. for rectal administration. Preferred dosage forms are those adapted for oral administration shaped as a tablet. They can be produced by conventional tableting techniques with conventional ingredients or excipients and with conventional tableting machines. A therapeutically effective amount of compound A ranges from about 50 mg to about 1000 mg per one-, two-, three-, four-, or five-unit dosage forms, preferably about 50 mg to 500 mg, about 100 to 400 mg, about 150 to 300 mg, about 200 mg, about 100 or 150 mg, about 150 to 200 mg, about 200 to 250 mg, about 250 to 300 mg, about 300 to 350 mg, or about 350 to 400 mg. The therapeutically effective amount of compound A may be administered per one- or two- or three-unit dosage forms on a daily basis. Preferably the effective amount of compound A per tablet is between 20 and 200 mg, 20 and 150 mg, 20 and 100 mg, or 50 and 100 mg.

The therapeutically effective amount of compound A may be administered one-time a day, or twice a day. The therapeutically effective amount of compound A may be administered daily on a continuous 28-day cycle. The therapeutically effective amount of compound A may be administered daily on a continuous 21-day cycle.

In order to facilitate the swallowing of such a dosage form by a mammal, it is advantageous to give the dosage form, in particular tablets, an appropriate shape. Tablets that can be swallowed comfortably are therefore preferably elongated rather than round in shape. Especially preferred are biconvex oblate tablets. As discussed hereunder in more detail, a film coat on the tablet further contributes to the ease with which it can be swallowed.

Tablets that give an immediate release of compound A upon oral ingestion and that have good bioavailability are designed in such a manner that the tablets disintegrate rapidly in the stomach (immediate release) and that the particles which are liberated thereby are kept away from one another so that they do not coalesce, give local high concentrations of compound A and the chance that the drug precipitates (bioavailability). The desired effect can be obtained by distributing said particles homogeneously throughout a mixture of a disintegrant and a diluent.

The formulations of the invention, in particular the tablets, may include one or more conventional excipients (pharmaceutically acceptable carrier) such as disintegrants; diluents; fillers; binders; wetting agents, surfactants or surface-active carriers; buffering agents; lubricants; glidants; thickening agents; sweetening agents; flavors; colors; and coating material excipients. Some excipients can serve multiple purposes.

Suitable disintegrants are those that have a large coefficient of expansion. Examples thereof are hydrophilic, insoluble or poorly water-soluble crosslinked polymers such as crospovidone (crosslinked polyvinylpyrrolidone) and croscarmellose (crosslinked sodium carboxymethylcellulose). The amount of disintegrant in immediate release tablets according to the present invention may conveniently range from about 3 to about 15% (w/w) and preferably is about 7 to 9% (w/w). These amounts tend to be larger than usual in tablets in order to ensure that the particles are spread over a large volume of the stomach contents upon ingestion. Because disintegrants by their nature yield sustained release formulations when employed in bulk, it is advantageous to dilute them with an inert substance called a diluent or filler.

A variety of materials may be used as diluents or fillers. Examples are spray-dried or anhydrous lactose, sucrose, dextrose, mannitol, sorbitol, starch, cellulose (e.g. micro-crystalline cellulose Avicel™), dihydrated or anhydrous dibasic calcium phosphate, and others known in the art, and mixtures thereof. Preferred is a commercial spray-dried mixture of lactose monohydrate (75%) with microcrystalline cellulose (25%) which is commercially available as Microcela™. The amount of diluent or filler in the tablets may conveniently range from about 20% to about 70% (w/w) and preferably ranges from about 25% to about 60% (w/w). Preferred is microcrystalline cellulose and silicified microcrystalline cellulose.

Lubricants and glidants can be employed in the manufacture of certain dosage forms, and will usually be employed when producing tablets. Examples of lubricants and glidants are hydrogenated vegetable oils, e.g., sodium stearyl fumarate, hydrogenated Cottonseed oil, magnesium stearate, stearic acid, sodium lauryl sulfate, magnesium lauryl sulfate, colloidal silica, talc, mixtures thereof, and others known in the art. Interesting lubricants and glidants are magnesium stearate, and mixtures of magnesium stearate with colloidal silica. A preferred lubricant is magnesium stearate. A preferred glidant is colloidal anhydrous silica. A preferred lubricant is hydrogenated vegetable oil type I, most preferably hydrogenated, deodorized Cottonseed oil (commercially available from Karlshamns as Akofine NF™ (formerly called Sterotex™)). Glidants generally comprise 0.2 to 7.0% of the total tablet weight, in particular 0.5 to 1.5%, more in particular 1 to 1.5% (w/w).

Lubricants generally comprise 0.2 to 7.0% of the total tablet weight, in particular 0.2 to 1%, more in particular 0.5 to 1% (w/w).

Other excipients such as coloring agents and pigments may also be added to the tablets of the present invention. Coloring agents and pigments include titanium dioxide and dyes suitable for food. A coloring agent is an optional ingredient in the tablet of the present invention, but when used the coloring agent can be present in an amount up to 3.5% based on the total tablet weight.

Flavors are optional in the composition and may be chosen from synthetic flavor oils and flavoring aromatics or natural oils, extracts from plants leaves, flowers, fruits and so forth and combinations thereof. These may include cinnamon oil, oil of wintergreen, peppermint oils, bay oil, anise oil, eucalyptus, thyme oil. Also useful as flavors are vanilla, citrus oil, including lemon, orange, grape, lime and grapefruit, and fruit essences, including apple, banana, pear, peach, strawberry, raspberry, cherry, plum, pineapple, apricot and so forth. The amount of flavor may depend on a number of factors including the organoleptic effect desired. Generally, the flavor will be present in an amount from about 0% to about 3% (w/w).

Tablets of the present invention may further be film-coated to improve taste, to provide ease of swallowing and an elegant appearance. Many suitable polymeric film-coating materials are known in the art. A preferred film-coating material is hydroxypropyl methylcellulose HPMC, especially HPMC 2910 5 mPa·s. Other suitable film-forming polymers also may be used herein, including, hydroxypropylcellulose, and acrylate-methacrylate copolymers. Besides a film-forming polymer, the film coat may further comprise a plasticizer (e.g. propylene glycol) and optionally a pigment (e.g. titanium dioxide). The film-coating suspension also may contain talc as an anti-adhesive. In immediate release tablets, the film coat is small and in terms of weight accounts for less than about 3% (w/w) of the total tablet weight. Alternatively, enteric coatings may also be employed.

As known in the art, tablet blends may be dry-granulated or wet-granulated before tableting, with the use of binders. The tableting process itself is otherwise standard and readily practiced by forming a tablet from desired blend or mixture of ingredients into the appropriate shape using a conventional tablet press, or by roller compaction.

Tablets comprising amorphous solid dispersions of compound A may also be prepared by 3D printing. The filaments for 3D printing may be prepared by hot melt extrusion at for example 150° C. with 10%, 20% or 30% w/w of compound A using the orally pharmaceutically acceptable polymer as described herein.

Formulations for oral use may also be presented as hard gelatin or HPMC capsules wherein the particles presented herein are mixed with an inert solid diluent, for example, calcium carbonate, calcium phosphate or kaolin.

### Methods of Treatments and Medical Uses

The pharmaceutical formulations described herein may be administered in any of the herein disclosed dosage forms and regimens or by means of those dosage forms and regimens established in the art whenever use of the pharmaceutical formulation is required for a subject in need thereof.

The pharmaceutical formulations and dosage forms of the present invention are useful in methods for treating, ameliorating and/or preventing a disease, a syndrome, a condition that is affected by the inhibition of MALT1.

One embodiment of the present invention is directed to a method of treating a MALT1-dependent or MALT1-mediated disease or condition in a subject in need thereof, including an animal, a mammal, and a human in need of such treatment, comprising administering to the subject a therapeutically effective amount of a pharmaceutical formulation or dosage form described herein.

The MALT1-dependent or MALT1-mediated disease or condition may be selected from cancers of hematopoietic origin or solid tumors such as chronic myelogenous leukemia, myeloid leukemia, non-Hodgkin lymphoma (NHL), NF-_{κ}B-driven B cell malignancies, and other B cell lymphomas.

Cancers that may benefit from a treatment with pharmaceutical formulations and dosage forms described herein include, but are not limited to, lymphomas, leukemias, carcinomas, and sarcomas, e.g. non-Hodgkin's lymphoma (NHL (including B-cell NHL)), diffuse large B-cell lymphoma (DLBCL), mantle cell lymphoma (MCL), follicular lymphoma (FL), mucosa-associated lymphoid tissue (MALT) lymphoma, marginal zone lymphoma (MZL), T-cell lymphoma, Hodgkin's lymphoma, Burkitt's lymphoma, multiple myeloma, chronic lymphocytic leukemia (CLL), small lymphocytic lymphoma (SLL), Waldenstrom macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

In an alternate embodiment, the disorder or condition is selected from non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenstrom macroglobulinemia.

In yet another embodiment of the invention, the disorder or condition is lymphoma. In another embodiment of the invention, the disorder or condition is the activated B cell like (ABC) subtype of diffuse large B-cell lymphoma (DLBCL). In another embodiment of the invention, the disorder or condition is germinal center B cell like (GCB) subtype of diffuse large B-cell lymphoma (DLBCL). in another embodiment of the invention, the disorder or condition is non-germinal center B cell like (non-GCB) subtype of diffuse large B-cell lymphoma (DLBCL).

In an additional embodiment of the invention, the disorder or condition is chronic lymphocytic leukemia (CLL). In another embodiment, the disorder or condition small lymphocytic lymphoma (SLL).

In another embodiment of the invention, the lymphoma is MALT lymphoma.

In another embodiment of the invention, the disorder or condition is Waldenstrom macroglobulinemia (WM).

In yet another embodiment, the disorder or condition is selected from the group consisting of diffuse large B-cell lymphoma (DLBCL), mantle cell lymphoma (MCL), follicular lymphoma (FL), and mucosa-associated lymphoid tissue (MALT) lymphoma.

In an alternate embodiment, the disorder or condition is non-Hodgkin's lymphoma (NHL). In a further embodiment, the non-Hodgkin's lymphoma (NHL) is B-cell NHL.

In yet another embodiment, the disorder or condition is primary and secondary central nervous system lymphoma, transformed follicular lymphoma, or API2-MALT1 fusion dependent disease.

In another embodiment of the invention, the disorder or condition (cancer or immunological disease (such as any of the cancers listed above)) is relapsed or refractory to prior treatment.

In another embodiment of the invention, the disorder or condition is cancer (such as any of the cancers mentioned above) and the subject has received prior treatment with a Bruton tyrosine kinase inhibitor (BTKi).

In an alternate embodiment of the invention, the disorder or condition is cancer (such as any of the cancers mentioned above) and the subject is relapsed or refractory to prior treatment with a Bruton tyrosine kinase inhibitor (BTKi).

In particular, pharmaceutical formulations and dosage forms of the invention are useful for treating or ameliorating diseases, syndromes, conditions, or disorders such as diffuse large B-cell lymphoma (DLBCL), mantle cell lymphoma (MCL), follicular lymphoma (FL), mucosa-associated lymphoid tissue (MALT) lymphoma, marginal zone lymphoma, chronic lymphocytic leukemia (CLL) including 17p-depleted CLL, small lymphocytic lymphoma (SLL), and Waldenstrom macroglobulinemia (WM). In particular, pharmaceutical formulations and dosage forms of the invention are useful for treating or ameliorating DLBCL tumors with CD79A/B or CARD11 mutations, including tumors with acquired resistance to ibrutinib (BTK, PLCγ2 or CARD11 mutations), ibrutinib resistant CLL/MCL/WM tumors and MALT lymphoma (MALT translocation). Pharmaceutical formulations and dosage forms of the invention are also useful for treating or ameliorating diffuse large B-cell lymphoma, activated B cell-like subtype (ABC-DLBCL).

Pharmaceutical formulations and dosage forms of the invention may be used for the treatment of a subject that is relapsed or refractory to a prior treatment. This prior treatment may be a treatment with a Bruton tyrosine kinase inhibitor (BTKi) like ibrutinib. Particular cohorts of patients suitable for treatment with the pharmaceutical formulations and dosage forms of the invention include: i) relapsed and refractory patients with CLL, MCL, or WM following ibrutinib progression; ii) relapsed and refractory DLBCL patients; iii) relapsed and refractory patients with indolent NHL such as FL or MZL.

Pharmaceutical formulations and dosage forms of the invention may be used for the treatment of immunological diseases including, but not limited to, autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

The pharmaceutical formulations described herein may be employed in combination with one or more other medicinal agents, more particularly with other anti-cancer agents, e.g. chemotherapeutic, anti-proliferative or immunomodulating agents, particularly a BTK inhibitor such as ibrutinib, AC0058 (ACEA Therapeutics, Inc.), AS-0871, AS-1763, AS-550 (Carna Biosciences, Inc.), BIIB068, BIIB091 (Biogen, Inc.), BMS-986142 (Bristol-Myers Squibb Company), zanubrutinib, acalabrutinib, CG-806 (Aptose Biosciences Inc.), CGI1746 (Gilead Sciences), CX-1440 (Huadong Medicine Co., Ltd.), DTRMWXHS-12 (Zhejiang DTRM Biopharma Co. Ltd.), evobrutinib, GDC-0834 (Roche Holding AG), HCl-1401 (Elevar Therapeutics, Inc.), ICP-022 (InnoCare), LOU064 (Novartis AG), LOXO-305 (Eli Lilly and Company), LY3337641 (Eli Lilly and Company), M7583 (Merck KGaA), MK-1026 (ArQule, Inc.), NRX0492 (Nurix Therapeutics, Inc.), PRN1008, PRN473 (Principia Biopharma, Inc.), RG7845 (Roche Holding AG), RN486 (Roche Holding AG), SAR442168 (Sanofi), SN1011 (SinoMab BioScience Limited), spebrutinib, TAK020 (Takeda Pharmaceutical Company Limited), TG-1701, TG-1702 (TG Therapeutics, Inc.), tirabrutinib, TP-4207 (Sumitomo Dainippon Pharma Co., Ltd.), vecabrutinib; or with adjuvants in cancer therapy, e.g. immunosuppressive or anti-inflammatory agents.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All possible combinations of the above-indicated embodiments are considered to be embraced within the scope of this invention.

Reference is now made to the following examples, which illustrate the invention in a non-limiting fashion.

### EXAMPLES

### Abbreviations and Definitions

BLD Bend lab dryer
BSV/TSV Bulk and tapped specific volume, inverse of bulk and tapped density
DSC Differential scanning calorimetry
HPMCAS hydroxypropylmethylcellulose-acetate succinate
HPMC-E5 E5-grade of hydroxypropylmethylcellulose

### LOQ Limit Of Quantification

MALT1 Mucosa-Associated lymphoid tissue Lymphoma Translocation protein 1

### NA Not Applicable

PSD-1 Pharmaceutical Spray Dryer with 100 kg/hr drying gas capacity
XRD X-ray diffraction

### RH Relative Humidity

SDD Spray-dried dispersion
SEM Scanning electron microscopy
Tg Glass transition temperature
UPLC Ultra performance liquid chromatography

### Example 1: Preparation of crystalline 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A) hydrate Form I

Compound A hydrate Form I was prepared by analogy to the synthesis method as described in Example 158 of WO 2018/119036. The compound prepared by this method was confirmed to be a hydrate crystalline form. The crystalline hydrate was characterized by XRPD. Table 1 provides peak listings and relative intensities for the XPRD.

TABLE 1

| Pos. [°2Th.] | Rel. Int. [%] |
|---|---|
| 3.3492 | 39.37 |
| 6.6640 | 4.90 |
| 8.3921 | 99.18 |
| 9.5561 | 2.00 |
| 9.9822 | 17.19 |
| 10.4253 | 1.40 |
| 10.7270 | 21.94 |
| 12.0003 | 10.48 |
| 12.2582 | 8.63 |
| 12.6973 | 75.08 |
| 13.3111 | 100.00 |
| 13.5391 | 25.04 |
| 14.0837 | 34.93 |
| 14.5855 | 33.39 |
| 15.3831 | 8.76 |
| 15.5724 | 12.24 |
| 15.9676 | 9.12 |
| 16.7336 | 64.64 |
| 17.4857 | 6.14 |
| 18.0702 | 31.51 |
| 18.3862 | 8.90 |
| 19.2183 | 16.27 |
| 20.0081 | 39.14 |
| 20.3419 | 26.48 |
| 21.1256 | 34.24 |
| 21.3242 | 15.79 |
| 22.0092 | 35.62 |
| 22.5028 | 16.08 |
| 23.1445 | 7.75 |
| 23.4107 | 11.70 |
| 23.8241 | 9.17 |
| 24.3918 | 19.32 |
| 24.5913 | 18.26 |
| 24.9140 | 46.75 |
| 25.3974 | 32.79 |
| 25.5768 | 43.71 |
| 26.1570 | 11.50 |
| 26.7323 | 3.55 |
| 27.2280 | 21.80 |
| 27.5416 | 32.47 |
| 27.8348 | 16.14 |
| 28.0704 | 8.75 |
| 28.6818 | 11.22 |
| 29.3712 | 4.98 |
| 30.3808 | 4.04 |
| 31.2917 | 10.24 |
| 31.5862 | 11.98 |
| 32.9442 | 5.01 |
| 33.6350 | 4.99 |
| 33.9874 | 2.68 |
| 34.4781 | 3.01 |
| 34.8120 | 4.21 |
| 35.6513 | 3.06 |
| 37.1454 | 3.83 |
| 38.9841 | 1.18 |
| 39.4671 | 1.81 |
| 40.6150 | 4.58 |
| 42.5268 | 2.93 |
| 43.4580 | 2.63 |
| 44.1621 | 1.20 |
| 45.6961 | 2.04 |
| 46.7044 | 4.03 |
| 48.7494 | 8.95 |
| 48.8885 | 4.57 |
| 49.8753 | 4.63 |

### Example 2: Preparation of Crystalline Compound a Monohydrate Form III, Seed Material

Approximately 200 mg of Compound A hydrate Form I obtained by Example 1 was added to 400-800 μL of either ethyl acetate or isopropyl acetate and the resulting suspension stirred at 60° C. for 5 days. The precipitate was then filtered and dried under vacuum at 50° C. for 24 hours to yield crystalline monohydrate Form I of Compound A.

### Example 3: Preparation of Crystalline Compound a Monohydrate Form III

1-(1-oxo-1,2-dihydroisoquinolin-5-yl)-5-(trifluoromethyl)-N-[2-(trifluoromethyl)pyridin-4-yl]-1H-pyrazole-4-carboxamide (100 g) obtained by a procedure analogous to the synthesis method as described in Example 158 of WO 2018/119036 was charged in a flask (R1) together with ethanol (150-170 mL) and ethyl acetate (80-100 mL). The obtained mixture was heated to 40-50° C. and stirred for 0.5-2 hours. Water (4-7 mL) was then added and the water content was measured by Karl Fischer titration. The content of R1 was warmed to 40-55° C. and filtered into a second flask (R2) pre-heated at 40-55° C. R1 was rinsed with ethyl acetate (80-100 mL) at 40-50° C. and the content filtered into R2. n-Heptane (340-410 mL) was charged into R2 in about 20-40 min. maintaining 40-55° C. The obtained solution was seeded with 1.9-2.1 g of crystalline monohydrate of Compound A and the obtained mixture was stirred at 40-55° C. for 4-8 hours. n-heptane (680-750 mL) was added in 10-15 hours maintaining 40-55° C.; the obtained mixture was stirred for additional 2-5 hours at 40-55° C., then it was cooled down to 20-25° C. for 7-13 hours. The suspension was stirred at 20-25° C. for 12-18 h, then it was filtered and washed with n-heptane (180-250 mL). After drying under vacuum at 45-55° C. for 15-22 hours, crystalline 1-(1-oxo-1,2-dihydroisoquinolin-5-yl)-5-(trifluoromethyl)-N-[2-(trifluoromethyl)pyridin-4-yl]-1H-pyrazole-4-carboxamide monohydrate Form III was obtained with an 80% yield.

### Example 4: Preparation of Crystalline Compound a Monohydrate Form III

1-(1-oxo-1,2-dihydroisoquinolin-5-yl)-5-(trifluoromethyl)-N-[2-(trifluoromethyl)pyridin-4-yl]-1H-pyrazole-4-carboxamide monohydrate (25 g) obtained by a procedure analogous to the synthesis method as described in Example 158 of WO 2018/119036 was charged in a flask (R1) together with water (2.5-4.5 mL) and isopropyl alcohol (IPA) (100 mL). The obtained mixture was heated to 50° C. and stirred for 0.5-2 hours. n-Heptane (125 mL) was charged into R1. The obtained solution was seeded with 500 mg of crystalline monohydrate of Compound A and the obtained mixture was stirred at 50° C. for 72 hours. n-Heptane (275 mL) was added in 12 hours maintaining 50° C.; the obtained mixture was stirred for additional 58 hours at 50° C., then it was cooled down to 20-25° C. for 2 hours. The suspension was stirred at 20-25° C. for 94 h, then it was filtered and washed with n-heptane (100 mL). After drying under vacuum at 50° C. for 24 hours, crystalline 1-(1-oxo-1,2-dihydroisoquinolin-5-yl)-5-(trifluoromethyl)-N-[2-(trifluoromethyl)pyridin-4-yl]-1H-pyrazol e-4-carboxamide monohydrate was obtained with a 90% yield.

The crystalline monohydrate Form III was characterized by XRPD. Table 2 provides peak listings and relative intensities for the XPRD.

TABLE 2

| Pos. [°2Th.] | Rel. Int. [%] |
|---|---|
| 8.2904 | 25.26 |
| 8.6250 | 23.96 |
| 9.3485 | 2.24 |
| 11.4511 | 14.20 |
| 12.5682 | 4.31 |
| 13.6202 | 45.95 |
| 13.9754 | 21.49 |
| 15.4397 | 41.22 |
| 15.8867 | 3.10 |
| 16.4426 | 100.00 |
| 16.6283 | 17.71 |
| 17.5110 | 14.58 |
| 17.9121 | 41.41 |
| 18.6250 | 4.18 |
| 19.6673 | 14.48 |
| 21.5675 | 11.28 |
| 21.9258 | 14.96 |
| 22.1775 | 15.69 |
| 22.5940 | 41.75 |
| 23.6809 | 85.80 |
| 24.0437 | 15.69 |
| 24.5412 | 27.75 |
| 25.1642 | 29.90 |
| 25.7310 | 49.96 |
| 27.1482 | 38.49 |
| 27.6772 | 10.70 |
| 27.9857 | 5.32 |
| 29.0996 | 7.66 |
| 29.3985 | 10.88 |
| 29.9267 | 20.17 |
| 30.9874 | 5.22 |
| 31.8056 | 12.06 |
| 32.8799 | 7.23 |
| 33.1991 | 5.73 |
| 34.4861 | 6.97 |
| 36.3854 | 7.95 |
| 36.6246 | 4.89 |
| 37.3258 | 7.90 |
| 37.8748 | 7.87 |
| 38.3143 | 5.55 |
| 40.8261 | 2.60 |
| 42.4567 | 3.57 |
| 43.2056 | 2.48 |
| 43.7464 | 4.48 |
| 45.0366 | 1.28 |
| 46.0177 | 2.48 |
| 48.3545 | 1.47 |

### Example 5: Manufacture of Amorphous Solid Dispersions of Compound a by Solvent Evaporation

Seven different amorphous solid dispersions of compound A and the orally pharmaceutically acceptable polymer were prepared in a 96-well plate by a solvent evaporation method. The seven orally pharmaceutically acceptable polymers were:
Eudragit L100-55 (Poly(methacrylic acid, ethyl acrylate) 1:1);
HPMCAS (Dow) (Hydroxypropyl methylcellulose acetate succinate Affinisol™ 716);
HPMCAS (Dow) (Hydroxypropyl methylcellulose acetate succinate Affinisol™ 912);
HPMCAS (Dow) (Hydroxypropyl methylcellulose acetate succinate Affinisol™ 126);
HPMC E5 (Hydroxypropyl methylcellulose/Hypromellose/HPMC 2910, 5 mPa.$);
PVP VA64 (Polyvinylpyrrolidone-vinyl acetate copolymer/Copovidone/Kollidon VA64);
HPMC E5/SLS (Sodium lauryl sulfate/Sodium dodecyl sulfate/SDS).

Starting material compound A (crystalline monohydrate Form III) and the orally pharmaceutically acceptable polymer were both dissolved in a mixture of dichloromethane and methanol (50/50, v/v) or ethanol. Mixtures were prepared using an automated liquid handling workstation (Hamilton Microlab STAR plus). After dispensing, amorphous drug-polymer films were generated by rapid evaporation of the organic solvent. This was achieved by evaporation under reduced pressure for one hour using a vacuum oven set at 70° C. and 200 mbar. The resulting films (12 replicates of each formulation) were cooled down after which a physical stability assessment was performed by cross-polarized imaging. As reference, a Compound A-only concept was included in the study. In function of the stability assessment, reference plates were prepared in a similar way. These films did not contain the compound A, only the corresponding polymers.

### Example 6: Physical Stability Assay of the Amorphous Solid Dispersions of Compound A, Prepared by Solvent Evaporation According to Example 5

Films were evaluated for their physical stability. This was done by stressing the films for 4 weeks at 40° C./75% relative humidity (RH). A crystallinity assessment was performed by cross-polarized imaging prior to (to) and after 4 weeks stressing (ti). No crystalline material was detected after film casting (to) or after 4 weeks storage at 40° C./75% RH (ti), for all compound A/polymer ratios.

### Example 7: Dissolution Study of the Amorphous Solid Dispersions of Compound A, Prepared by Solvent Evaporation According to Example 5

In vitro, 2-phase (SGF/FaSSIF) miniaturized dissolution was performed, where the amount of dissolved compound A was monitored in function of time. Before starting the dissolution assay, the films were stored for one day at room temperature. By doing so, most of the residual solvent had evaporated. Actual dissolution experiments were performed in 96 1 mL glass vials using the Hamilton STAR plus liquid handling platform for both sampling and sample preparation. Before adding the dissolution media to the films, an equilibration time of approximately 60 minutes was needed to preheat the media to 37° C. After equilibration, 300 μL of preheated SGF (37° C., pH 1.3) was added to the amorphous solid dispersions. After 15 minutes of incubation in SGF, 600 μL of preheated concentrated FaSSIF (37° C., pH 10.5) was added to the samples. The addition of this concentrated FaSSIF to SGF resulted in a medium with a similar composition to the typical FaSSIF medium (pH 6.5) used in 1-phase dissolution studies. At predetermined time intervals, aliquots were withdrawn from the dissolution media and filtered through a 0.45 μm GHP membrane filter. Subsequently, the filtered solutions were quantitatively diluted (10×) with N-methylpyrrolidone (NMP) to prevent possible precipitation. The amount of dissolved compound A was determined by UPLC. Films were incubated at 37° C. throughout the entire assay. Experiments were performed in duplicate.

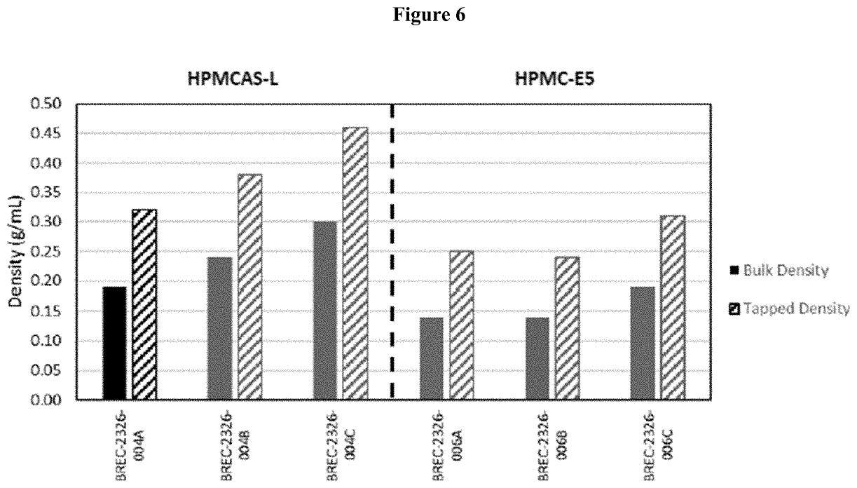

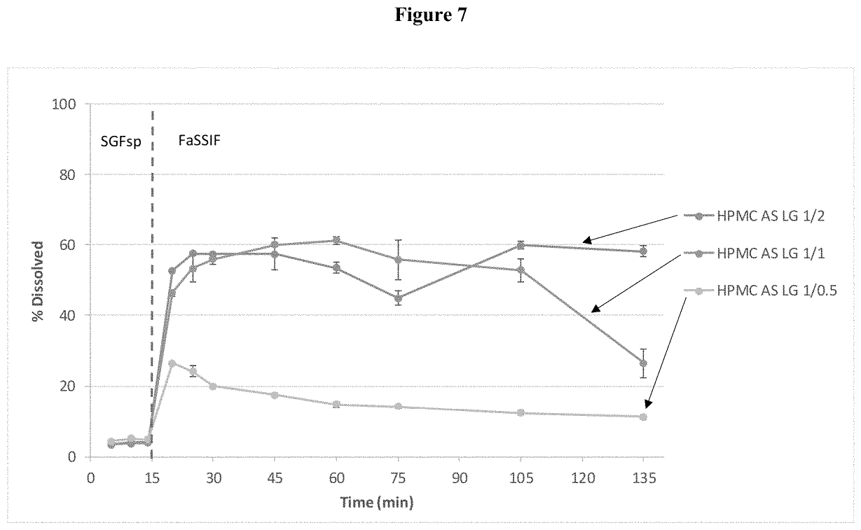

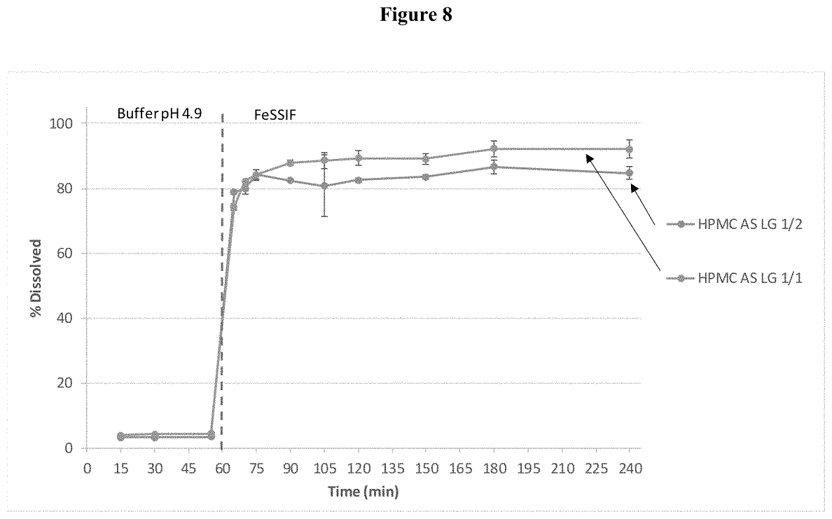

FIGS. 6 to 8 show the dissolution profiles in SGF-FaSSIF. For the neat amorphous Compound A reference, an initial release of 30% to 40% of the total amount of Compound A present in the films was measured (60-80 μg/mL). Most polymers showed similar dissolution behaviour to the neat amorphous Compound A. Only Eudragit L100-55 and HPMC in combination with SLS seemed to increase the initial dissolution rate and give a slightly higher release after 2 hours. Observations were similar for all Compound A/polymer ratios (1/3, 1/1 and 2/1).

### Example 8: Manufacture of Amorphous Solid Dispersions of Compound a by Spray-Drying

A total of six prototype compound A spray-dried dispersions formulations were manufactured on a modified Pharmaceutical Spray Dryer with 100 kg/hr drying gas capacity. This unit was capable of exceeding the nominal gas flow-rate range listed by the manufacturer, and could be equipped with a 6′ chamber extension to extend particle residence times inside the drying chamber. The solid starting materials, including compound A monohydrate crystalline Form III, were dissolved into a suitable spray solvent and atomized at a high pressure through a small orifice nozzle to generate small droplets that were rapidly dried with hot nitrogen gas. The resulting particles were collected using a 6″ diameter cyclone into a collection container, and then placed in a convection tray dryer to remove residual solvent remaining from the spray drying process.

The formulations varied active content from 33.3-66.7 wt % solids and the cellulosic dispersion polymer, either HPMCAS or HPMC-E5. The spray solvent was varied based on the polymer type, as HPMC-E5 required approximately 20% water to dissolve in primarily acetone solvent. The spray solids content was varied from 6.7-12.7 wt % based on the solution viscosity data in an attempt to match particle size between active loadings in the same formulation. This determination was performed by first considering the Lefebvre droplet size model with an adjustment based on the solids loading (higher solids loading leads to larger particles at the same droplet size).

TABLE 3
Manufacturing summary of prototype Compound
A/HPMCAS spray-dried dispersions

| | 33.3/66.7 | 50.0/50.0 | 66.7/33.3 |
|---|---|---|---|
| | Compound A/ | Compound A/ | Compound A/ |
| Formulation | HPMCAS-LG | HPMCAS-LG | HPMCAS-LG |
| Lot No. | BREC- | BREC- | BREC- |
| | 2326-004A | 2326-004B | 2326-004C |
| Batch Size (g) | 451 | 451 | 451 |
| Solvent (w/w) | 100% Acetone | | |
| Atomizer | Pressure | | |
| Drying Gas Flow | 1849 | 1849 | 1849 |
| Rate (g/min) | | | |
| Solution Flow | 197 | 195 | 191 |
| Rate (g/min) | | | |
| Atomization | 300 | | |
| Pressure (psig) | | | |
| Inlet Temper- | 121 | 115 | 113 |
| ature (° C.) | | | |
| Outlet Temper- | 38 | 35 | 36 |
| ature (° C.) | | | |
| Solids Content | 8.4 | 10.0 | 12.7 |
| (wt %) | (2.8 API, | (5.0 API, | (8.5 API, |
| | 5.6 Polymer) | 5.0 Polymer) | 4.2 Polymer) |
| Calculated | 7.1 | 5.7 | 4.2 |
| Viscosity (cP) | | | |
| Wet Yield (%) | 92 | 86 | 83 |
| Dry Yield (%) | 86 | 80 | 76 |
| Secondary Drying | Convection Tray Dryer: 40-50° C. for 23-75 hr | | |
| Conditions | | | |
| Residual | 0.02 | 0.12 | 0.02 |
| Acetone (wt %) | | | |

TABLE 4
Manufacturing summary of prototype
Compound A/HPMC-E5 spray-dried dispersions

| | 33.3/66.7 | 50.0/50.0 | 66.7/33.3 |
|---|---|---|---|
| | Compound A/ | Compound A/ | Compound A/ |
| Formulation | HPMC-E5 | HPMC-E5 | HPMC-E5 |
| Lot No. | BREC- | BREC- | BREC- |
| | 2326-006A | 2326-006B | 2326-006C |
| Batch Size (g) | 500 | 501 | 500 |
| Solvent (w/w) | 80/20 Acetone/Water | | |
| Atomizer | Pressure | | |
| Drying Gas Flow | 1848 | 1849 | 1848 |
| Rate (g/min) | | | |
| Solution Flow | 126 | 123 | 122 |
| Rate (g/min) | | | |
| Atomization | 300 | | |
| Pressure (psig) | | | |
| Inlet Temper- | 139 | 139 | 135 |
| ature (° C.) | | | |
| Outlet Temper- | 46 | 45 | 46 |
| ature (° C.) | | | |
| Solids Content | 6.7 | 8.0 | 10.2 |
| (wt %) | (2.2 API, | (4.0 API, | (6.8 API, |
| | 4.5 Polymer) | 4.0 Polymer) | 3.4 Polymer) |
| Calculated | 12.3 | 9.5 | 6.9 |
| Viscosity (cP) | | | |
| Wet Yield (%) | 95 | 92 | 90 |
| Dry Yield (%) | 90 | 88 | 86 |
| Secondary Drying | Convection Tray Dryer: 40-50° C. for 23-75 hr | | |
| Conditions | | | |
| Residual | <LOQ | 0.04 | <LOQ |
| Acetone (wt %) | | | |

The percentages and solid contents of compound A in Tables 3 and 4 are provided for the monohydrate form III. The person skilled in the art will correct the amounts for compound A, with a 1.0386 hydrate correction factor.

### Example 9: Particle Size Distribution Analysis of Amorphous Spray-Dried Dispersions, as Prepared According to Example 8

Particle size distribution analysis was performed using a Malvern Mastersizer 3000 using an AeroS dispersion unit. A dispersive air pressure of 3 bar was used for all measurements using the Fraunhofer approximation.

TABLE 5
Tabulated particle size distribution results for the prototype amorphous sprayed-dried dispersions

| | | Dv10 | Dv50 | Dv90 | D[3,2] | D[4,3] | |
|---|---|---|---|---|---|---|---|
| Sample Description | Lot No. | μm | μm | μm | μm | μm | Span |
| 33.3/66.7 Compound A/ | BREC-2326-004A | 11.1 | 38.7 | 80.5 | 15.8 | 42.9 | 1.8 |
| HPMCAS-LG | | | | | | | |
| 50.0/50.0 Compound A/ | BREC-2326-004B | 10.0 | 33.1 | 69.8 | 13.1 | 37.4 | 1.8 |
| HPMCAS-LG | | | | | | | |
| 66.7/33.3 Compound A/ | BREC-2326-004C | 8.1 | 28.0 | 59.5 | 10.1 | 31.4 | 1.8 |
| HPMCAS-LG | | | | | | | |
| 33.3/66.7 Compound A/ | BREC-2326-006A | 9.7 | 31.4 | 66.7 | 14.0 | 36.5 | 1.8 |
| HPMC E5 | | | | | | | |
| 50.0/50.0 Compound A/ | BREC-2326-006B | 9.9 | 32.0 | 68.6 | 13.1 | 36.6 | 1.8 |
| HPMC E5 | | | | | | | |
| 66.7/33.3 Compound A/ | BREC-2326-006C | 8.3 | 27.5 | 60.1 | 11.1 | 31.6 | 1.9 |
| HPMC E5 | | | | | | | |

### Example 10: Solubility of Amorphous SDD of Compound A/HPMCAS-LG (1:2), Lot No. BREC-2326-004A, as Prepared According to Example 8

Solubility was evaluated in pH 2 gastric media (0.01N HCl), pH 6.5 phosphate buffered saline

(PBS) intestinal media with 0.0%, 0.5% and 1.0% simulated intestinal fluid (SIF) bile salt micelles, at 90 minutes and 24 hours. Samples were dosed at 2.5 mg/mL and placed on a rocker table within a warm box at 37° C. which gently agitated samples over the duration of the test. After ultracentrifugation the drug concentration was measured by HPLC.

TABLE 6
Tabulated Amorphous Solubility in Biorelevant Media
for the 33.3/66.7 Compound A/HPMCAS-LG SDD

| | Concentration (μg/mL) | | Cᵤₗₜᵣₐ/C_{ub} | K_{mic} |
|---|---|---|---|---|
| Media | 90 minutes | 1 Day | 1 Day | 1 Day |
| 0.01N HCl (pH 2) | 46 | 58 | — | — |
| PBS (pH 6.5) | 127 | 12 | 1.0 | 6109 |
| 0.5% SIF in PBS (pH 6.5) | 743 | 648 | 52.8 | |
| 1.0% SIF in PBS (pH 6.5) | 945 | 1587 | 129.3 | |

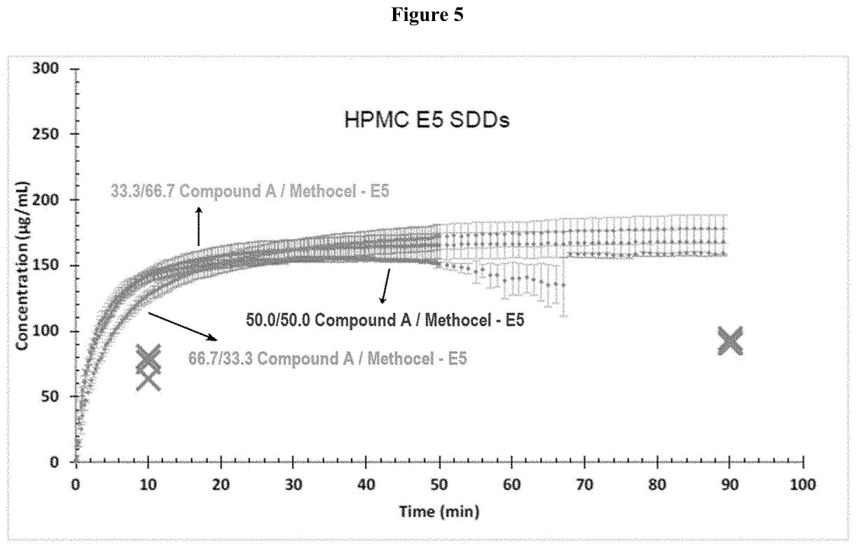

In FIG. 5 it is shown the Amorphous Solubility in Biorelevant Media for the 33.3/66.7 Compound A/HPMCAS-LG SDD (lot no. BREC-2326-004A).

### Example 11: Dissolution Performance of Compound A/HPMCAS-LG Amorphous SDDs (1:2, 1:1, and 2:1), and of Compound A/HPMC-E5 Amorphous SDDs (1:2, 1:1, and 2:1), as Prepared According to Example 8

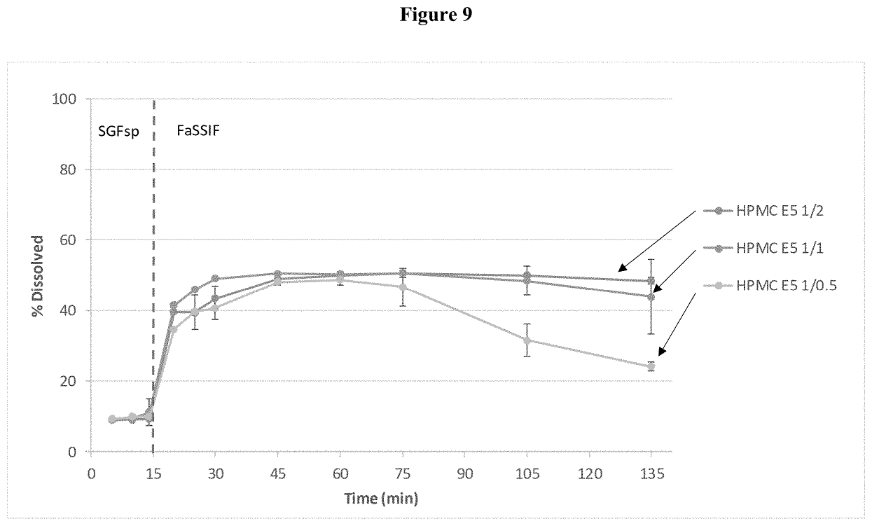

Dissolution rate decreases with increasing drug loading in HPMCAS-LG amorphous SDDs (FIG. 9). In terms of sustainment of supersaturation, all three HPMCAS-LG amorphous SDD are equivalent within the 90 minutes of the test. The ultracentrifuge samples confirm a sustained drug concentration of ca. 110 μg/mL at 90 minutes for all three HPMCAS-LG amorphous SDDs.

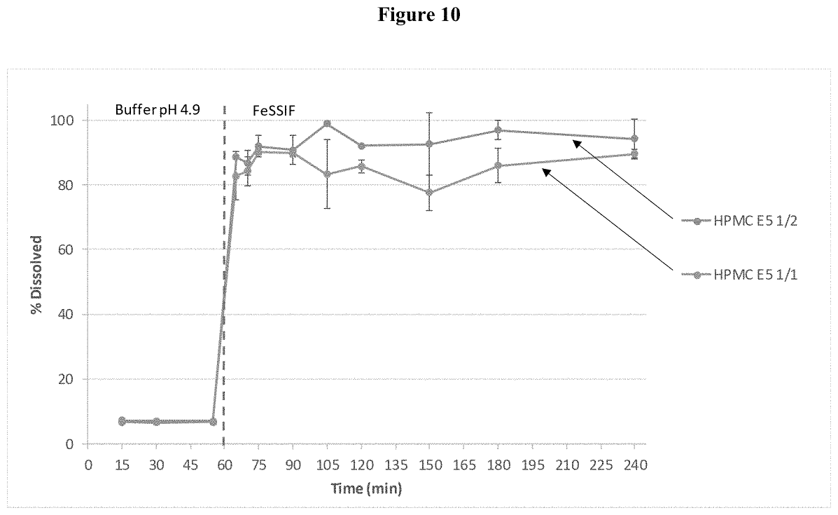

The dissolution rate is relatively independent of active loading for HPMC E5 amorphous SDDs (FIG. 10). The HPMC E5 SDD provide a slightly lower level of supersaturation relative to the HPMCAS-LG amorphous SDDs (89-94 vs 106-110 μg/mL) with sustainment throughout the 90-minute test.

The Pion UV-probe dissolution apparatus was used to conduct non-sink dissolution testing to determine the relative performance for the six compound A amorphous SDDs. Ultracentrifuge samples were also collected at 10 and 90 minutes and analyzed by HPLC to confirm actual concentration of dissolved drug at those time points.

TABLE 7
Dissolution performance, FIG. 9

| Amorphous | | Ultra 10 min | Ultra 90 min |
|---|---|---|---|
| SDD Formulation | Lot No. | (μg/mL) | (μg/mL) |
| 33.3/66.7, Compound A/ | BREC- | 106 ± 5 | 106 ± 1 |
| HPMCAS-LG | 2326-004A | | |
| 50/50, Compound A/ | BREC- | 106 ± 2 | 110 ± 1 |
| HPMCAS-LG | 2326-004B | | |
| 66.7/33.3, Compound A/ | BREC- | 75 ± 5 | 110 ± 0 |
| HPMCAS-LG | 2326-004C | | |

TABLE 8
Dissolution performance, FIG. 10

| Amorphous | | Ultra 10 min | Ultra 90 min |
|---|---|---|---|
| SDD Formulation | Lot No. | (μg/mL) | (μg/mL) |
| 33.3/66.7, Compound | BREC-2326- | 76 ± 2 | 89 ± 1 |
| A/HPMC-E5 | 006A | | |
| 50/50, Compound | BREC-2326- | 80 ± 8 | 94 ± 1 |
| A/HPMC-E5 | 006B | | |
| 66.7/33.3, Compound | BREC-2326- | 63 ± 6 | 92 ± 2 |
| A/HPMC-E5 | 006C | | |

### Example 12: Powder Density of Amorphous Sprayed-Dried Dispersions, as Prepared According to Example 8

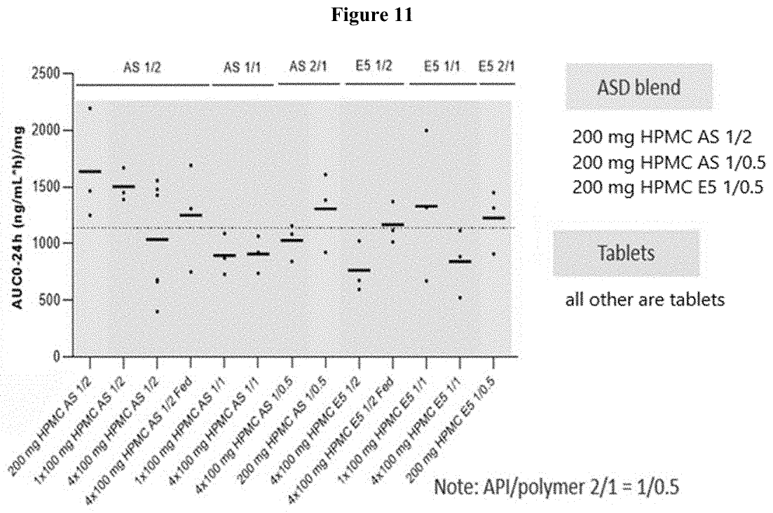

Powder bulk and tapped density was measured by repeatedly tapping a bed of powder contained in a 10 mL graduated cylinder. Bulk and tapped density results are shown in FIG. 11, with tabulated results including flowability metrics (Carr index and Hausner ratio) given in Table 9.

TABLE 9
Tabulated powder density results for the prototype
compound A amorphous SDD formulations

| | | Bulk | Tapped | Carr | |
|---|---|---|---|---|---|
| | | Density | Density | Index | Hausner |
| Description | Lot No. | (g/mL) | (g/mL) | (%) | Ratio |
| 33.3/66.7 Compound A/HPMCAS-LG | BREC-2326-004A | 0.19 | 0.32 | 39 | 1.64 |
| 50/50 Compound A/HPMCAS-LG | BREC-2326-004B | 0.24 | 0.38 | 37 | 1.58 |
| 66.7/33.3 Compound A/HPMCAS-LG | BREC-2326-004C | 0.30 | 0.46 | 35 | 1.54 |
| 33.3/66.7 Compound A/HPMC E5 | BREC-2326-006A | 0.14 | 0.25 | 44 | 1.81 |
| 50/50 Compound A/HPMC E5 | BREC-2326-006B | 0.14 | 0.24 | 41 | 1.70 |
| 66.7/33.3 Compound A/HPMC E5 | BREC-2326-006C | 0.19 | 0.31 | 41 | 1.70 |

### Example 13: Thermal Analysis by DSC of Amorphous Sprayed-Dried Dispersions, as Prepared According to Example 8

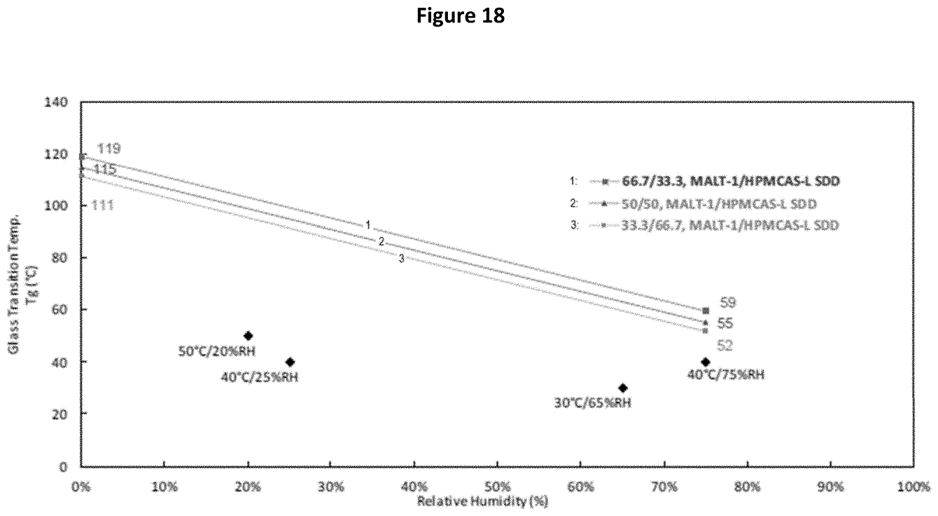

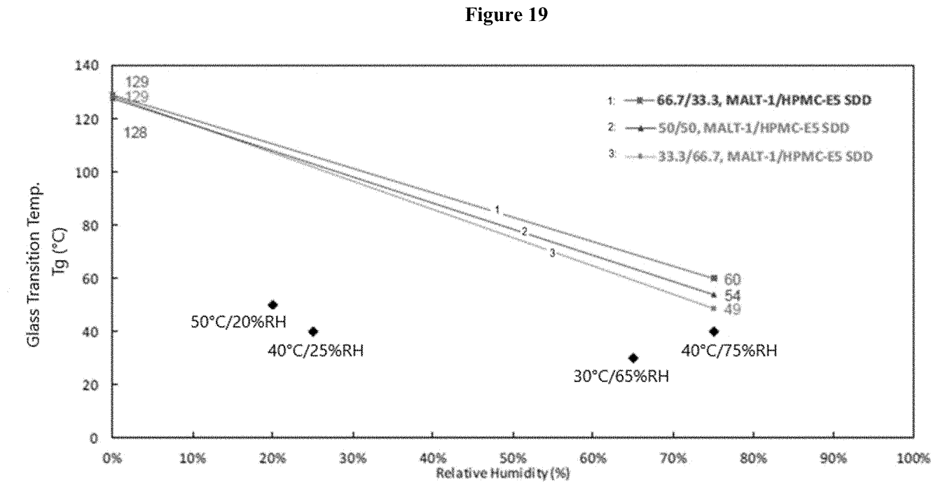

The Tg vs RH results for HPMCAS-LG amorphous SDDs (FIG. 18 and Table 10) show the Tg's range from 111 to 119° C. under dry conditions and are depressed down to 52 to 59° C. at 75% RH. Both dry and wet Tg increase with increased drug loading. The results in FIG. 19 and Table 11 show that relative to the HPMCAS-LG amorphous SDDs, the HPMC E5 amorphous SDDs have a higher Tg under dry conditions (129° C.) and similar Tg at 75% RH (49° C. to 60° C.). Altogether these data show that Tg is higher than 40° C. at the most stringent storage conditions (40° C./75 RH), and display therefore a low risk of recrystallization, demonstrating thereby an optimal physical stability.

TABLE 10
DSC Tg vs % RH results for Compound A/HPMCAS-LG amorphous SDDs.

| Amorphous SDD | | Tg < 5% RH | Tg 75% RH |
|---|---|---|---|
| formulation | Lot No. | (° C.) | (° C.) |
| 33.3/66.7 Compound A/ | BREC-2326-004A | 111 | 52 |
| HPMCAS-LG SDD | | | |
| 50/50 Compound A/ | BREC-2326-004B | 115 | 55 |
| HPMCAS-LG SDD | | | |
| 66.7/33.3 Compound A/ | BREC-2326-004C | 119 | 59 |
| HPMCAS-LG SDD | | | |

TABLE 11
DSC Tg vs % RH results for Compound A/HPMC E5 amorphous SDDs.

| | | Tg < 5% RH | Tg 75% RH |
|---|---|---|---|
| Amorphous SDD formulation | Lot No. | (° C.) | (° C.) |
| 33.3/66.7 Compound A/HPMC E5 SDD | BREC-2326-006A | 129 | 49 |
| 50/50 Compound A/HPMC E5 SDD | BREC-2326-006B | 128 | 54 |
| 66.7/33.3 Compound A/HPMC E5 SDD | BREC-2326-006C | 129 | 60 |

### Example 14: Potency of the Amorphous Sprayed-Dried Dispersions, as Prepared According to Example 8

The potency was measured by use of a HPLC method with the following details:

### Instrument: Agilent 1200 HPLC

### Column: Agilent Poroshell 120 EC-C18, 3×50 mm, 2.7 μm

### Column Temperature: 30° C.

### Autosampler Temperature: Ambient

Mobile Phase A: 95/5 10 mM Ammonium Acetate (aq)/ACN

### Mobile Phase B: ACN

HPLC Gradient

| Time(min) | A(% Vol) | B(% Vol) |
|---|---|---|
| 0.0 | 65 | 35 |
| 3.0 | 65 | 35 |
| 3.1 | 5 | 95 |
| 5.0 | 5 | 95 |
| 5.1 | 65 | 35 |
| 6.0 | 65 | 35 |

Flow Rate: 1.0 mL/min
Run Time: 6 minutes
Working concentration: 0.25 mgA/mL

### Sample Diluent: 7/3, ACN/Water

### Injection Volume: 10 μL

### Detection: 250 nm

The measured potencies were as follows:

| | | Potency | Range | % of Target |
|---|---|---|---|---|
| SDD Formulation: | Lot No. | (mgA/g) | (n = 2) | Potency |
| 33.3/66.7, Compound A/HPMCAS-LG SDD | BREC-2326-004A | 325 | 0.1 | 97.6 |
| 50/50, Compound A/HPMCAS-LG SDD | BREC-2326-004B | 490 | 5 | 98.0 |
| 66.7/33.3, Compound A/HPMCAS-LG SDD | BREC-2326-004C | 661 | 10 | 99.1 |
| 33.3/66.7, Compound A/HPMC E5 SDD | BREC-2326-006A | 335 | 4 | 100.5 |
| 50/50, Compound A/HPMC E5 SDD | BREC-2326-006B | 506 | 2 | 101.2 |
| 66.7/33.3, Compound A/HPMC E5 SDD | BREC-2326-006C | 674 | 1 | 101.1 |

### Example 15: Compositions of Six 100 mg Uncoated Tablets with a 30% Load of an Amorphous Solid Dispersion of Compound A

| | Spray Dried Powder: Compound A/Polymer Ratio | | | | | |
|---|---|---|---|---|---|---|
| | 1/2 | | 1/1 | | 2/1 | |
| | mg/tablet | % w/w | mg/tablet | % w/w | mg/tablet | % w/w |
| Spray Dried Powder of | 300.00 | 30.00 | 200.00 | 30.00 | 150.00 | 30.00 |
| Example 8 | | | | | | |
| (HPMCAS-LG or HPMC E5) | | | | | | |
| Microcrystalline cellulose | 367.50 | 36.75 | 245.00 | 36.75 | 183.75 | 36.75 |
| (Avicel PH-101) | | | | | | |
| Croscarmellose Sodium | 25.00 | 2.50 | 16.67 | 2.50 | 12.50 | 2.50 |
| (Ac-Di-Sol SD-711) | | | | | | |
| Silica, Colloidal Anhydrous | 5.00 | 0.50 | 3.33 | 0.50 | 2.50 | 0.50 |
| (Aerosil 200) | | | | | | |
| Magnesium Stearate | 2.50 | 0.25 | 1.67 | 0.25 | 1.25 | 0.25 |
| (Ligamed MF-2-V) | | | | | | |
| Total lntragranular | 700.00 | 70.00 | 466.67 | 70.00 | 350.00 | 70.00 |
| Silicified Microcrystalline | 262.50 | 26.25 | 175.00 | 26.25 | 131.25 | 26.25 |
| cellulose | | | | | | |
| (Prosolv SMCC HD 90) | | | | | | |
| Croscarmellose Sodium | 25.00 | 2.50 | 16.67 | 2.50 | 12.50 | 2.50 |
| (Ac-Di-Sol SD-711) | | | | | | |
| Silica, Colloidal Anhydrous | 5.00 | 0.50 | 3.33 | 0.50 | 2.50 | 0.50 |
| (Aerosil 200) | | | | | | |
| Magnesium Stearate | 7.50 | 0.75 | 5.00 | 0.75 | 3.75 | 0.75 |
| (Ligamed MF-2-V) | | | | | | |
| Total Extragranular | 300.00 | 30.00 | 200.00 | 30.00 | 150.00 | 30.00 |
| Core Tablet | 1000.00 | 100.00 | 666.67 | 100.00 | 500.00 | 100.00 |

The Spray Dried Powder contains Compound A and the polymer (HPMCAS-LG or HPMC E5) in the ratio as indicated in the Table above. This Spray Dried Powder comprising compound A in the 6 uncoated tablets above, was prepared according to Example 8.

### Example 16: Composition of Compound a Eq. 100 mg Oral Film-Coated Tablets

| Component | Quantity per Unit (mg) |
|---|---|
| Compound A eq. 100 mg oral | 1000.00 |
| tablet (½) of Example 15 | |
| Opadry II 85F250050 Pink^{a} | 30.00 |
| Purified water^{b} | <q. s .^{b, c}> |
| Total film coated tablet | 1030.00 |

^{a}Qualitative composition of Coating powder Opadry II 85F250050 Pink is given in the next table.
^{b}Removed during processing.
^{c}Typical coating suspension contains approximately 20 wt % solids.

Qualitative composition of coating
powder pink Opadry II 85F250050
Component
Polyvinyl alcohol-part hydrolyzed
Titanium dioxide
Macrogol/PEG
Talc
Iron oxide red

A coating suspension was prepared by dispersing coating powder in purified water until a suspension was obtained. The core tablets were transferred into a suitable coating pan. The coating solution was then sprayed upon the core tablets using the film coating technique. The film coated tablets were dried, after spraying, in the same coating pan. The coated tablets were collected and packaged in a suitable container.

### Example 17: Preparation of Pure Amorphous Form of Compound a (without Polymer) with Buchi Method

First a solution of 10 wt. % Compound A was prepared by weighing off 20 g Compound A (hydrate form III), added to 180 g pure Acetone wider stirring, and stirred until fully dissolved. The solution was then spray dried on a Buchi spray drier and post dried in a vacuum tray dryer using the operating conditions in the table below. The spray dried Compound A was found to be amorphous by XRPD and measured to have a glass transition temperature of 132° C.

| Formulation | Pure API |
|---|---|
| Batch Size (g) | 20 |
| Solvent (w/w) | 100% Acetone |
| Atomizer | 2-fluid |
| Drying Gas Flow Rate (kg/h) | 30-35 |
| Solution Flow Rate (g/min) | ~8 |
| Atomization Gas Flow Rate (mm) | 30 |
| Inlet Temperature (° C.) | 74 |
| Outlet Temperature (° C.) | 54 |
| Solids Content (wt %) | 10 |
| Secondary Drying Conditions | Vacuum Tray Dryer: |
| | 30° C./300 |
| | mBar/Nitrogen |
| | flow for 19 hr |
| Residual Acetone (wt %) | N/A |

### Example 18: Compositions of Four Capsules with 3 Amorphous Sprayed-Dried

dispersions of compound A, and one capsule with amorphous Compound A

| Formulation 18.1 | mg/capsule | % w/w |
|---|---|---|
| Spray Dried Powder Com- | 66.67 | 35.00 |
| pound A/HPMCAS-LG 2/1 | | |
| Aerosil (silica) | 9.52 | 5.00 |
| Avicel (MCC) | 114.29 | 60.00 |
| Total | 190.49 | 100.00 |

| Formulation 18.2 | mg/capsule | % w/w |
|---|---|---|
| Spray Dried Powder Com- | 66.67 | 35.00 |
| pound A/HPMC E5 2/1 | | |
| Aerosil (silica) | 9.52 | 5.00 |
| Avicel (MCC) | 114.29 | 60.00 |
| Total | 190.49 | 100.00 |

| Formulation 18.3 | mg/capsule | % w/w |
|---|---|---|
| Spray Dried Powder Compound | 40.00 | 35.00 |
| A/HPMCAS-LG ½ | | |
| Aerosil (silica) | 5.71 | 5.00 |
| Avicel (MCC) | 68.58 | 60.00 |
| Total | 114.29 | 100.00 |

| Formulation 18.4, prepared | | |
|---|---|---|
| according to Example 17 | mg/capsule | % w/w |
| Amorphous Compound A | 100.00 | 35.00 |
| Aerosil (silica) | 14.29 | 5.00 |
| Avicel ® (MCC) | 171.43 | 60.00 |
| Total | 285.71 | 100.00 |

The amounts of compound A were calculated based on the anhydrous form of compound A.

### Example 19: Stability Results of Prepared Amorphous Formulations of Compound A

Stability of the prepared formulations of Compound A was measured by XRD at normal and accelerated conditions, showing that all the prepared formulations of the compound, be it alone or combined with a polymer, are amorphous.

### XRD Results

| | Neat | API/ | API/ | API/ | API/ | API/ | API/ | API/ | API/ | API/ |
|---|---|---|---|---|---|---|---|---|---|---|
| | API | HPMCAS | HPMCAS | HPMCAS | EL-100- | EL-100- | EL-100- | HPMCAS | HPMCAS | HPMCAS |
| | DRY | MG | MG | MG | 55 | 55 | 55 | E5 | E5 | E5 |
| ratio | NA | 1/3 | 1/1 | 1/0,5 | 1/3 | 1/1 | 1/0,5 | 1/3 | 1/1 | 1/0,5 |
| API/polymer | | | | | | | | | | |
| Conditions | | | | | | | | | | |
| T0 | amorph. | amorph. | amorph. | amorph. | amorph. | amorph. | amorph. | amorph. | amorph. | amorph. |
| RT closed 1M | amorph. | amorph. | amorph. | amorph. | amorph. | amorph. | amorph. | amorph. | amorph. | amorph. |
| RT/56% RH | amorph. | amorph. | amorph. | amorph. | amorph. | amorph. | amorph. | amorph. | amorph. | amorph. |
| open 1M | | | | | | | | | | |
| 40° C. closed 1M | amorph. | amorph. | amorph. | amorph. | amorph. | amorph. | amorph. | amorph. | amorph. | amorph. |
| 40° C/75% RH | amorph. | amorph. | amorph. | amorph. | amorph. | amorph. | amorph. | amorph. | amorph. | amorph. |
| open 1M | | | | | | | | | | |
| 50° C. closed 1M | amorph. | amorph. | amorph. | amorph. | amorph. | amorph. | amorph. | amorph. | amorph. | amorph. |
| RT closed 2M | amorph. | amorph. | amorph. | amorph. | amorph. | amorph. | amorph. | amorph. | amorph. | amorph. |
| RT/56% RH | amorph. | amorph. | amorph. | amorph. | amorph. | amorph. | amorph. | amorph. | amorph. | amorph. |
| open 2M | | | | | | | | | | |
| 40° C. closed 2M | amorph. | amorph. | amorph. | amorph. | amorph. | amorph. | amorph. | amorph. | amorph. | amorph. |
| 40° C/75% RH | amorph. | amorph. | amorph. | amorph. | amorph. | amorph. | amorph. | amorph. | amorph. | amorph. |
| open 2M | | | | | | | | | | |
| 50° C. closed 2M | amorph. | amorph. | amorph. | amorph. | amorph. | amorph. | amorph. | amorph. | amorph. | amorph. |
| RT closed 6M | amorph. | amorph. | amorph. | amorph. | amorph. | amorph. | amorph. | amorph. | amorph. | amorph. |
| RT/56% RH | amorph. | amorph. | amorph. | amorph. | amorph. | amorph. | amorph. | amorph. | amorph. | amorph. |
| open 6M | | | | | | | | | | |
| 40° C. closed 6M | amorph. | amorph. | amorph. | amorph. | amorph. | amorph. | amorph. | amorph. | amorph. | amorph. |
| 40° C./75% RH | amorph. | amorph. | amorph. | amorph. | amorph. | amorph. | amorph. | amorph. | amorph. | amorph. |
| open 6M | | | | | | | | | | |
| 50° C. closed 6M | amorph. | amorph. | amorph. | amorph. | amorph. | amorph. | amorph. | amorph. | amorph. | amorph. |

Neat API: Active Pharmaceutical Ingredient, which is Compound A
HPMCAS MG: from Shin-Etsu Chemical Co., Ltd
EL-100-55: Eudragit ® L 100-55
1M: 1 month
2M: 2 months
6M: 6 months
amorph.: amorphous

### Example 20: Compositions of 65 and 80 mg Uncoated Tablets with a 30% Load of an Amorphous Solid Dispersion of Compound A

| | 65 mg | | 80 mg | |
|---|---|---|---|---|
| | mg/tablet | % w/w | mg/tablet | % w/w |
| Spray Dried Dispersion Powder of Example 8 | 195.000 | 30.00 | 240.00 | 30.00 |
| 33.3/66.7 Compound A/HPMCAS-LG | | | | |
| Microcrystalline cellulose (Avicel PH-101) | 238.875 | 36.75 | 294.00 | 36.75 |
| Croscarmellose Sodium (Ac-Di-Sol SD-711) | 16.250 | 2.50 | 20.00 | 2.50 |
| Silica, Colloidal Anhydrous (Aerosil 200) | 3.250 | 0.50 | 4.00 | 0.50 |
| Magnesium Stearate (Ligamed MF-2-V) | 1.625 | 0.25 | 2.00 | 0.25 |
| Total Intragranular | 455.000 | 70.00 | 560.00 | 70.00 |
| Silicified Microcrystalline cellulose | 170.625 | 26.25 | 210.00 | 26.25 |
| (Prosolv SMCC HD 90) | | | | |
| Croscarmellose Sodium (Ac-Di-Sol SD-711) | 16.250 | 2.50 | 20.00 | 2.50 |
| Silica, Colloidal Anhydrous (Aerosil 200) | 3.250 | 0.50 | 4.00 | 0.50 |
| Magnesium Stearate (Ligamed MF-2-V) | 4.875 | 0.75 | 6.00 | 0.75 |
| Total Extragranular | 195.000 | 30.00 | 240.00 | 30.00 |
| Core Tablet | 650.000 | 100.00 | 800.00 | 100.00 |

### Example 21: Composition of Compound a Eq. 65 mg Oral Film-Coated Tablets

| Component | Quantity per Unit (mg) |
|---|---|
| Compound A eq. 65 mg | 650.00 |
| oral tablet of Example 20 | |
| Opadry II 85F250050 Pink^{a} | 19.50 |
| Purified water^{b} | <q. s.^{b, c}> |
| Total film coated tablet | 669.50 |

^{a}Qualitative composition of Coating powder Opadry II 85F250050 Pink is given in the table of Example 16.
^{b}Removed during processing.
^{c}Typical coating suspension contains approximately 20 wt % solids.

### Example 22: Composition of Compound a Eq. 80 mg Oral Film-Coated Tablets

| Component | Quantity per Unit (mg) |
|---|---|
| Compound A eq. 80 mg | 800.00 |
| oral tablet of Example 20 | |
| Opadry II 85F250050 Pink^{a} | 24.00 |
| Purified water^{b} | <q. s.^{b, c}> |
| Total film coated tablet | 824.00 |

^{a}Qualitative composition of Coating powder Opadry II 85F250050 Pink is given in the table of Example 16.
^{b}Removed during processing.
^{c}Typical coating suspension contains approximately 20 wt % solids.

A coating suspension was prepared by dispersing coating powder in purified water until a suspension was obtained. The core tablets were transferred into a suitable coating pan. The coating solution was then sprayed upon the core tablets using the film coating technique. The film coated tablets were dried, after spraying, in the same coating pan. The coated tablets were collected and packaged in a suitable container.

### Example 23: Preliminary PK Results in Healthy Participants

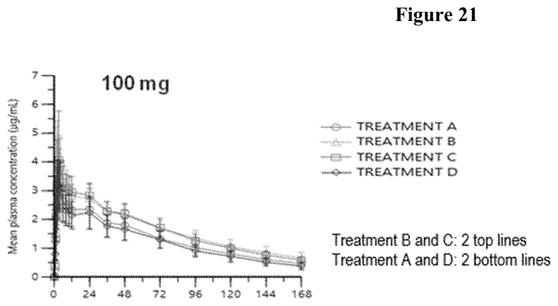

FIG. 21 shows the preliminary PK results in healthy participants. The date is also summarized in the table below.

| PK | | Geometric LSM | | | Geometric mean | | |
|---|---|---|---|---|---|---|---|
| Parameter | Dose | Treat- | | Geometric | ratios (%) | | |
| (unit) | (mg) | ment | N | mean | Comparison | PE | 90% CI |
| Cₘₐₓ | 100 | A | 10 | 3.32 | | | |
| (μg/mL) | | B | 10 | 4.55 | B vs A | 137 | 112-167 |
| | | C | 10 | 4.47 | C vs A | 135 | 110-164 |
| | | D | 10 | 3.22 | D vs A | 97 | 79-118 |

‘PE’ means point estimate
‘90% CI’ means 90% confidence interval
Treatment A (reference capsule): 100 mg Compound A supplied as 2×50 mg LFHG PEG1500 capsules (as described in WO2020/169738); fasted (N=10)
Treatment B (test): 100 mg Compound A supplied as one 100 mg uncoated ASD tablet (Compound A/HPMCAS-LG ratio 1/2 as described in Example 15); fasted (N=10)
Treatment C (test): 100 mg Compound A supplied as one 100 mg uncoated ASD tablet (Compound A/HPMCAS-LG ratio 1/1 as described in Example 15); fasted (N=10)
Treatment D (test): 100 mg Compound A supplied as one 100 mg LFHG PEG1500 capsule (as described in WO2020/169738); fasted (N=10)
LFHG means liquid filled hard gelatin capsules

Based on the data, ASD tablets have a higher bioavailability than the LFHG PEG1500 capsules. The ASD tablets have a higher exposure than the PEG1500-based capsules.

1. Isolated, 1-(1-oxo-1,2-dihydroisoquinolin-5-yl)-5-(trifluoromethyl)-N-[2-(trifluoromethyl)pyridin-4-yl]-1H-pyrazole-4-carboxamide (compound A), or a pharmaceutically acceptable salt form thereof, in amorphous form or non-crystalline phase, wherein the amorphous form or non-crystalline phase of compound A is present in a weight percentage in respect of any crystalline form of compound A, of more than 90% w/w, preferably at least 95% w/w.

2. An amorphous solid dispersion comprising compound A, or a pharmaceutically acceptable salt form thereof; and an orally pharmaceutically acceptable polymer.

3. The amorphous solid dispersion of claim 2, wherein the weight-by-weight ratio of (compound A):(orally pharmaceutically acceptable polymer) is in the range of 5:1 to 1:5; preferably in the ratios of 5:1, 4:1, 3:1, 2:1, 1:1, 1:2, 1:3, 1:4, and 1:5.

4. The amorphous solid dispersion of claim 2 or 3, wherein the orally pharmaceutically acceptable polymer is a polymer used for spray-drying that has an apparent viscosity when dissolved at 20° C. of 1 to 5000 mPa·s, of 1 to 500 mPa·s, or of 1 to 100 mPa·s; or wherein the orally pharmaceutically acceptable polymer has an apparent viscosity in an organic solvent of 1 to 5000 mPa·s, of 1 to 500 mPa·s, or of 1 to 100 mPa; or wherein the orally pharmaceutically acceptable polymer is a polymer used for Hot Melt Extrusion and the molten polymer has an apparent viscosity of 1 to 1,000,000 Pa·s, of 100 to 100,000 Pa·s, or of 500 to 10,000 Pa·s.

5. The amorphous solid dispersion of claim 2 or 3, wherein the orally pharmaceutically acceptable polymer is selected from the group comprising:
alkylcelluloses such as methylcellulose;
hydroxyalkylcelluloses such as hydroxymethylcellulose, hydroxyethylcellulose, hydroxypropylcellulose and hydroxybutylcellulose;
hydroxyalkyl alkylcelluloses such as hydroxyethyl methylcellulose and hydroxypropyl methylcellulose;
carboxyalkylcelluloses such as carboxymethylcellulose;
alkali metal salts of carboxyalkylcelluloses such as sodium carboxymethylcellulose;
carboxyalkylalkylcelluloses such as carboxymethylethylcellulose;
carboxyalkylcellulose esters;
hydroxypropylmethylcellulose phthalate (HPMCP);
chitin derivates such as chitosan;
polysaccharides such as starches, pectines (sodium carboxymethylamylopectine), cyclodextrins or a derivative thereof, carrageenans, galactomannans, tragacanth, agar agar, gummi arabicum, guar gummi and xanthan gummi;
polyacrylic acids, olyacrylates, and the salts thereof;
polymethacrylic acids, polymethacrylates, the salts and esters thereof, methacrylate copolymers;
polyvinylalcohol (PVA), co-polymers of PVA (e.g., Kollicoat IR), crospovidone (PVP-CL), polvinylpyrrolidone-polyvinylacetate copolymer (PVP-PVA);
polyalkylene oxides such as polyethylene oxide and polypropylene oxide and copolymers of ethylene oxide and propylene oxide;
polymers of ethylene oxide or polyethylene glycols of molecular weights in the range of 1500-20000, particularly with MW of 4000-6000;
polyvinylpyrrolidone (PVP) of MW ranging from 2500 to 3000000;
Gelita® Collagel; or
any combination thereof;
and optionally a surface-active carrier.

6. The amorphous solid dispersion of claim 2 or 3, wherein the orally pharmaceutically acceptable polymer is HPMCAS, HPMC E5, Eudragit® E, Eudragit® L, PVP VA64, any combination thereof, and wherein the orally pharmaceutically acceptable polymer or combination thereof is optionally mixed with Sodium Lauryl Sulfate (SLS).

7. The amorphous solid dispersion of claim 6, wherein the HPMCAS is HPMCAS-LG, HPMCAS-MG, HPMCAS-HG, HPMCAS-LF, HPMCAS-MF, HPMCAS-HF, HPMCAS-LMP, HPMCAS-MMP, HPMCAS-HMP, Affinisol™ HPMCAS 716, Affinisol HPMCAS 912, or Affinisol HPMCAS 126.

8. The amorphous solid dispersion of claim 6, wherein the Eudragit® L is Eudragit® L 100-55.

9. The amorphous solid dispersion of claim 6, wherein the orally pharmaceutically acceptable polymer is HPMC E5 mixed with a surface-active carrier, preferably SLS.

10. A particle comprising the amorphous solid dispersion of any one of claims 2-9.

11. The particle of claim 10, wherein said particle has a volume weighted particle size distribution Dv50, as measured by a static light scattering instrument, of from about 20 μm to about 90 μm, preferably from about 25 μm to about 80 μm, more preferably from about 25 μm to about 65 μm.

12. The particle of claim 10, wherein said particle has a Dv10 of volume weighted particle size distribution from about 1 μm to about 15 μm; and the Dv90 of the volume weighted particle size distribution is from about 40 μm to about 200 μm.

13. The particle of any one of claims 10-12 further comprising a pharmaceutically acceptable carrier.

14. A particle comprising compound A of claim 1, or a pharmaceutically acceptable salt form thereof.

15. The particle of claim 14, wherein said particle has a volume weighted particle size distribution Dv50, as measured by a static light scattering instrument, of from about 1 μm to about 100 μm, preferably from about 5 μm to about 80 μm, more preferably from about 25 μm to about 75 μm.

16. The particle of claim 14, wherein said particle has a Dv10 of volume weighted particle size distribution from about 0.1 μm to about 15 μm; and the Dv90 of the volume weighted particle size distribution is from about 3 μm to about 250 μm.

17. The particle of any one of claims 14-16 further comprising a pharmaceutically acceptable carrier.

18. A pharmaceutical composition comprising a pharmaceutically acceptable carrier; and (i) a therapeutically effective amount of compound A of claim 1, or a pharmaceutically acceptable salt form thereof; (ii) a therapeutically effective amount of an amorphous solid dispersion according to any one of claims 2-9; (iii) a therapeutically effective amount of the particles according to any one of claims 10-13; or (iv) a therapeutically effective amount of the particles according to any one of claims 14-17.

19. The pharmaceutical composition of claim 18, wherein the composition is a solid oral dosage form.

20. The pharmaceutical composition of claim 19, wherein the composition is a tablet, capsule, sachet, pill, lozenge, caplet, capsule, sachet, or troche.

21. The pharmaceutical composition of claim 19, wherein the composition is a Core Tablet having the following composition:

| | Spray Dried Powder: Compound A/Polymer Ratio | | | | | |
|---|---|---|---|---|---|---|
| | 1/2 | | 1/1 | | 2/1 | |
| | mg/tablet | % w/w | mg/tablet | % w/w | mg/tablet | % w/w |
| Spray Dried Powder | 300.00 | 30.00 | 200.00 | 30.00 | 150.00 | 30.00 |
| comprising Compound A and | | | | | | |
| polymer X | | | | | | |
| Microcrystalline cellulose | 367.50 | 36.75 | 245.00 | 36.75 | 183.75 | 36.75 |
| Croscarmellose Sodium | 25.00 | 2.50 | 16.67 | 2.50 | 12.50 | 2.50 |
| Silica, Colloidal Anhydrous | 5.00 | 0.50 | 3.33 | 0.50 | 2.50 | 0.50 |
| Magnesium Stearate | 2.50 | 0.25 | 1.67 | 0.25 | 1.25 | 0.25 |
| Silicified Microcrystalline | 262.50 | 26.25 | 175.00 | 26.25 | 131.25 | 26.25 |
| cellulose | | | | | | |
| Croscarmellose Sodium | 25.00 | 2.50 | 16.67 | 2.50 | 12.50 | 2.50 |
| Silica, Colloidal Anhydrous | 5.00 | 0.50 | 3.33 | 0.50 | 2.50 | 0.50 |
| Magnesium Stearate | 7.50 | 0.75 | 5.00 | 0.75 | 3.75 | 0.75 |
| Core Tablet | 1000.00 | 100.00 | 666.67 | 100.00 | 500.00 | 100.00 |

wherein polymer X is HPMCAS-LG or HPMC E5; and
wherein the Core Tablet is optionally coated; preferably coated with coating powder pink Opadry II 85F250050.

22. The pharmaceutical composition of claim 20, wherein the composition is a tablet, wherein the pharmaceutically acceptable carrier comprises a disintegrant, a glidant, a lubricant, a diluent, optionally a wetting agent, optionally a binder, and optionally a coating material.

23. The pharmaceutical composition of claim 20, wherein the composition is a capsule or a sachet, optionally further comprising a diluent.

24. A process for preparing the amorphous solid dispersion of any one of claims 2-9, comprising the steps of:
a) blending compound A, or a pharmaceutically acceptable salt form thereof; with an orally pharmaceutically acceptable polymer;
b) extruding said blend at a temperature in the range of 20-300° C.

25. The process according to claim 24, further comprising preparing particles, said process further comprising the steps of:
c) grinding the extrudate, and
d) optionally sieving the particles.

26. A process for preparing the amorphous solid dispersion of any one of claims 2-9, comprising the steps of:
a) blending compound A, or a pharmaceutically acceptable salt form thereof; with an orally pharmaceutically acceptable polymer and a suitable solvent;
b) spray-drying said blend.

27. The process according to claim 26, wherein the suitable solvent is selected from: alcohols selected from methanol, ethanol, n-propanol, iso-propanol, and butanol; ketones selected from acetone, methyl ethyl ketone, and methyl iso-butyl ketone; esters selected from ethyl acetate, and propylacetate; acetonitrile; dichloromethane; toluene; 1,1,1-trichloroethane; dimethyl acetamide; dimethylsulfoxide; combinations thereof; a mixture of methanol and dichloromethane, 60:40 (w:w) or 50:50 (w:w); and a mixture of acetone and water 80:20 (w:w).

28. A process for preparing the particle of any one of claims 14-16, comprising the step of spray-drying a mixture of compound A, or a pharmaceutically acceptable salt form thereof;
and a suitable solvent.

29. A process for preparing the particle of claim 17, comprising the step of spray-drying a mixture of compound A, or a pharmaceutically acceptable salt form thereof; and a suitable solvent; onto the surface of a pharmaceutically acceptable bead.

30. The process according to any one of claims 28-29, wherein the suitable solvent is as defined in claim 27.

31. The process according to any one of claims 24-30, further comprising preparing tablets or capsules; said process further comprising blending a therapeutically effective amount of the material obtained from any one of claims 24-30, with pharmaceutically acceptable excipients; and compressing said blend into tablets or filling said blend into capsules.

32. The amorphous solid dispersion of any one of claims 2-9, wherein said amorphous solid dispersion is obtainable by melt-extruding a mixture comprising compound A, or a pharmaceutically acceptable salt form thereof; and an orally pharmaceutically acceptable polymer.

33. The particles of any one of claims 10-13, wherein said particles are obtainable by grinding the amorphous solid dispersion of claim 32, and optionally sieving the obtained particles.

34. The amorphous solid dispersion of any one of claims 2-9, or the particles of any one of claims 10-13, wherein said amorphous solid dispersion or particles are obtainable by spray-drying a mixture comprising compound A, or a pharmaceutically acceptable salt form thereof; an orally pharmaceutically acceptable polymer; and a suitable solvent.

35. Compound A of claim 1, or a pharmaceutically acceptable salt form thereof; or the particles of any one of claims 14-16, wherein said compound A or particles are obtainable by spray-drying a mixture comprising compound A, or a pharmaceutically acceptable salt form thereof; and a suitable solvent.

36. The compound A or the particles as obtainable according to claim 35, wherein the mixture is sprayed-dried onto the surface of pharmaceutically acceptable beads.

37. Compound A of claim 1, or a pharmaceutically acceptable salt form thereof; the amorphous solid dispersion of any one of claims 2-9; the particles of any one of 10-13; or the particles of any one of claims 14-17; for use in the treatment of a disease, syndrome, condition, or disorder in a subject in need thereof, wherein said disease, syndrome, condition, or disorder is affected by the inhibition of MALT1.

38. A method of treating a disease, syndrome, condition, or disorder, wherein said disease, syndrome, condition, or disorder is affected by the inhibition of MALT1, comprising administering to a subject in need thereof a therapeutically effective amount of: (i) compound A of claim 1; or a pharmaceutically acceptable salt form thereof; (ii) the amorphous solid dispersion of any one of claims 2-9; (iii) the particles of any one of 10-13; or (iv) the particles of any one of claims 14-17.

39. Use of (i) compound A of claim 1, or a pharmaceutically acceptable salt form thereof; (ii) the amorphous solid dispersion of any one of claims 2-9; (iii) the particles of any one of 10-13; or (iv) the particles of any one of claims 14-17; in the manufacture of a medicament for the treatment of a disease, syndrome, condition, or disorder affected by the inhibition of MALT1.
